(19) United States
(12) Patent Application Publication
(10) Pub. No.: US 2016/0074298 A1
(43) Pub. Date: Mar. 17, 2016

(54) POLYPHENOL/FLAVONOID COMPOSITIONS AND METHODS OF FORMULATING ORAL HYGIENIC PRODUCTS

(71) Applicant: VIZURI HEALTH SCIENCES LLC, Fairfax, VA (US)

(72) Inventors: Philip J. BIRBARA, West Hartford, CT (US); Jeffrey S. CUMMINGS, Waltham, MA (US); Karl GINTER, Beltsville, MD (US); Krishna RAMAN, Wilmington, DE (US)

(21) Appl. No.: 14/854,858

(22) Filed: Sep. 15, 2015

Related U.S. Application Data
(60) Provisional application No. 62/050,650, filed on Sep. 15, 2014.

(51) Int. Cl. A61K 8/49; A61K 8/35; C07D 311/30; A61Q 11/00

(52) U.S. Cl. CPC A61K 8/498; A61Q 11/00; A61K 8/35; C07D 311/30; A61K 2800/87; A61K 2800/92

(57) ABSTRACT

Microemulsions and soluble alkali metal salts of relatively insoluble aglycone polyphenols within oral hygienic products are disclosed for treating oral inflammatory disorders. The formulations can act as a bactericide or bacteriostat. The methods include the process associated with the formation of a high temperature polyphenol/surfactant concentrate, a nano-particulate precipitation process in the presence of a surfactant and the solubilization of relatively insoluble aglycone polyphenols/flavonoids by the formation of soluble alkali metal salts within alkaline oral compositions. Also disclosed are compositions that persist in the oral cavity, penetrate teeth, and facilitate or enhance the delivery of chemical elements and active ingredients to teeth for preventing or treating disorders and diseases of the oral cavity. Methods of use, and methods of making, the aforementioned compositions are also disclosed.

### CROSS REFERENCE TO RELATED APPLICATION

This application claims the benefit of U.S. Provisional Patent Application Ser. No. 62/050,650, filed Sep. 15, 2014, which is hereby incorporated by reference in its entirety.

### FIELD OF THE INVENTION

The present invention provides oral compositions for treating various common dental diseases such as dental caries, gingivitis and periodontitis. In addition, the invention provides compositions that remain in the oral cavity for prolonged periods of time compared with currently available orally-acceptable compositions used in the prevention or treatment of oral cavity disorders and diseases including those involving teeth and gingiva. Further, the invention provides compositions that penetrate teeth through cracks, dentinal tubules, caries, and the cementoenamel junction. Other compositions are provided that facilitate or enhance the delivery of calcium, zinc, and other elements to teeth for the prevention or treatment of various dental disorders and diseases. In addition, the invention provides methods of using, and methods of making, the compositions of the invention (as well as kits comprising the compositions).

The orally acceptable compositions can be present in various different forms such as a dentifrice, paste, gel, powder, mouth rinse, mouthwash, tooth hardener, medication, anti-calculus composition, film, slurry, injectable solution, gum and lozenge. The compositions of the present invention may be used, for example, as pharmaceuticals, medical devices, nutraceuticals, and cosmeceuticals.

More particularly, compositions and methods for improving the aqueous solubility of aglycone polyphenols are disclosed. Such compositions and methods utilize stable polyphenol concentrates, microemulsions, and alkali polyphenol salts. Also included are methods for inhibiting and/or stopping bacterial growth.

### BACKGROUND OF THE INVENTION

Individual flavonoids can vary greatly in their biological activity (or be inactive), both in terms of toxicity and effectiveness against microbes such as viruses and bacteria. Compositions comprising polyphenolic compounds have been reported to have a wide range of biological activities, such as anti-oxidant, anti-inflammatory, anti-bacterial and anti-viral activities.

Polyphenols, and in particular the relatively insoluble aglycone forms of the flavone and flavonol flavonoids, have anti-cariogenic properties. Several in vitro and in vivo studies have investigated the effects of these flavonoids against bacterial microorganisms including Streptococcus mutans and inflammatory infections.

All flavonoids, a subset of polyphenol compounds, have the same basic chemical structure, a three-ringed molecule with hydroxyl (OH) groups attached. Flavonoids have the following general molecular structure as noted below:

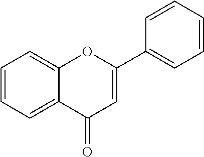 [SMILES: O=c1cc(-c2ccccc2)oc2ccccc12]

Flavonoids comprise approximately 5,000 naturally occurring compounds. A multitude of other substitutions can occur, giving rise to the many types of flavonoids, including the flavones (e.g., apigenin, luteolin, and so forth), flavonols (e.g., quercetin, myricetin, and so forth), flavonones (e.g., narigenin, hesperidin, and so forth), flavonols (or catechins) (e.g., epicatechin, gallocatechin, and so forth), anthocyanidins (e.g., cyanidin, pelargonidin, and so forth), and isoflavones (e.g., gunistein, daidezin, and so forth). Studies have demonstrated that flavones possess anti-oxidant, anti-mutagenic, anti-carcinogenic, anti-inflammatory, anti-proliferative, and anti-progression properties. (Patel, D, et al., Apigenin and cancer chemoprevention: Progress, potential, and promise, Intl. J. Oncology 2007 January; 30(1): 233-45).

Prior studies noted that aglycone flavonoids, in particular the flavone and flavonol components within propolis, a resinous bee product, were responsible for inhibiting the growth of oral microorganisms and associated activity of the enzyme glucosyltransferase (GTF). Several compounds, mainly polyphenols, have been identified in this natural product. Thirty compounds, including flavonoids, cinnamic acid derivatives, and terpenoids found in propolis, were tested for the ability to inhibit GTFs B, C, and D from Streptococcus mutans and GTF from S. sanguinis (GTF Ss). (Koo et al., Effects of Compounds Found in Propolis on Streptococcus Mutans Growth and on Glucosyltransferase Activity, Antimicrob Agents Chemother, 2002 May; 46(5): 1302-9.)

Koo et al. noted that flavones and flavonols were potent inhibitors of GTF activity in solution. Apigenin, a 4′,5,7-trihydroxyflavone, was the most effective inhibitor of GTFs both in solution (90.5 to 95% inhibition at a concentration of 135 microg/ml) and on the surface of sHA beads (30 to 60% at 135 μg/ml). Apigenin was also cited as a novel and most potent natural inhibitor of GTF activity. (Koo et. al., Effects of Compounds Found in Propolis on Streptococcus Mutans Growth and on Glucosyltransferase Activity, Antimicrob Agents Chemother, 2002 May; 46(5): 1302-9.)

U.S. Patent Application 2004/0057908 teaches an oral composition which includes an organoleptically suitable carrier and an amount of a terpenoid and a flavonoid, dispersed in the carrier, which is effective to prevent or treat dental caries, dental plaque formation, gingivitis, candidiasis, dental stomatitis, aphthous ulceration, or fungal infection. The invention also relates to various uses of oral compositions, containing a terpenoid, a flavonoid, or both, such uses include: inhibiting the activity of surface-bound glucosyltransferase; treating or inhibiting dental caries, gingivitis, candidiasis, and denture stomatitis; inhibiting the accumulation of microorganisms on an oral surface; and/or treating or inhibiting aphthous ulcerations on an oral surface.

Nearly all apigenin studies related to cancer and other research studies, including the cited studies of Koo et al, have utilized dimethyl sulfoxide (DMSO) and 100% ethyl alcohol as the solvent of choice due to the poor solubility of apigenin in water (0.003) milligram per milliliter (mg/ml)) as well as other aqueous and organic solvents suitable for oral compositions. (Li et al, Evaluation of Apigenin and [G-3H], Apigenin and analytical method development, J. of Pharmaceutical Sciences. Vol. 86, No. 6, June 1997). However, the use of apigenin vehicles containing DMSO and/or 100% ethyl alcohol is not suitable for human oral formulations.

Apigenin has been shown to be antifungal making it effective for treating denture stomatitis (Herrera et al., The Antifungal effect of 6 commercial extracts of Chilean propolis on Candida spp. Cien. Inv. Agr. 37 (1): 75-84 2010.). Also, it can act as an anti-inflammatory so it has also been investigated for periodontal disease. (Anti-inflammatory effects of apigenin on nicotine and lipopolysaccharide stimulated human periodontal ligament cells via heme oxygenase. November 2009 1374-1380 Vol. 9 Issue 12 Int Immunology). Apigenin is a strong antioxidant and was shown to inhibit oral carcinogenesis in hamsters. (S. Sylvan, Chemotherapeutic potential of apigenin in 7,12, dimethyl anthracene induced experimental oral carcinogenesis, Eur J pharmacol 2011 November 670 23).

Apigenin possesses anti-inflammatory activity in human periodontal ligament (hPDL) cells and works through a novel mechanism involving the action of heme oxygenase-1 (HO-1)1. Thus, apigenin has benefits as a host modulatory agent in the prevention and treatment of periodontal disease associated with smoking and dental plaque. (Gil-Saeng Jeong et al; Anti-inflammatory effects of apigenin on nicotine- and lipopolysaccharide-stimulated human periodontal ligament cells via heme oxygenase-1, International Immunopharmacology, Vol.: 9, November 2009).

U.S. Patent Application 2012/0213842 teaches methods of making and using flavonoids.

U.S. Pat. No. 8,637,569 relates to methods of increasing the solubility of poorly soluble compounds and methods of making and using formulations of such compounds.

U.S. Patent Application Ser. No. 61/886,977 teaches beverages containing polyphenols and methods of making same.

As background, hydrogen peroxide, in combination with sodium bicarbonate, can decompose rapidly. Peroxides, such as hydrogen peroxide, typically can break down in the presence of alkalinity, heat, light and/or metal ions as follows:

2H₂O₂→2H₂O+O₂ (gas)

In addition, sodium carbonate peroxide breaks down into sodium carbonate and hydrogen peroxide as follows:

2Na₂CO₃.3H₂O₂ (Sodium Carbonate peroxide)→2Na₂CO₃+3H₂O₂

The hydrogen peroxide acts as a potent oxidizing agent, and as an anti-microbial agent. Further the high alkalinity of sodium carbonate (˜pH=10.5) boosts the oxidizing effect of hydrogen peroxide.

Similarly, sodium bicarbonate can break down in the presence of hydrogen peroxide, heat and/or water as follows:

2NaHCO₃→Na₂CO₃+H₂O+CO₂ (gas)

Several clinical trials have demonstrated that sodium bicarbonate dentifrices have enhanced the plaque removal effectiveness of tooth brushing to a significantly greater extent than the non-sodium bicarbonate dentifrice products. (Putt, M. S et al, Enhancement of Plaque Removal Efficacy by Tooth Brushing with Baking Soda Dentifrices: Results of Five Clinical Studies, J. Clin Dent, 2008, 19(4); 11-9.) (Mankodi, S. et al, Evaluation of the Effects of Brushing on the Removal of Dental Plaque, J. Clin Dent, 1998; 9(3) 57-60.) (Drake, D. R., Enhanced Bactericidal Activity of Arm and Hammer Dental Care, Am J Dent, 1995, December; 8(6): 308-312.). Further, the topical application of the combination of hydrogen peroxide and sodium bicarbonate exhibited synergistic oxidative antimicrobial activity. (Miyasaki, K. T. et al, Antimicrobial Properties of Hydrogen Peroxide and Sodium Bicarbonate individually and in Combination Against Selected Oral, Gran-negative, Facultative Bacteria, J Dent Res, 1986, September 85 (9), 1142-1148)

The composition of several dentifrices containing significant concentrations of sodium bicarbonate and peroxide is considered to be noteworthy because of the difficulty of combining peroxide and baking soda in a way that avoids rapid decomposition due to interaction of the peroxide with the sodium bicarbonate when in solution. Several commercially available (e.g., Arm & Hammer PeroxiCare®) formulations use a combination of peroxide and sodium bicarbonate with slightly more than a 1% water concentration. The commercially available formulations utilize combinations of PEG-8 and a PEG/PPG 116/66 copolymer for stabilizing the peroxide and sodium bicarbonate ingredients.

Thus, there is a need for creating oral formulations and methods containing stable aglycone flavonoid concentrates, microemulsions and alkali aglycone flavonoid salts for treating oral inflammatory disorders and inhibiting bacterial growth in mammals. Also, there remains a need for orally-acceptable compositions that are not only highly effective in combating the onset and progression of dental-associated disorders and diseases but also persist in the oral cavity longer than currently available prophylactic and therapeutic dentifrices, pastes, gels, powders, mouth rinses, mouthwashes, tooth hardeners, medications, anti-calculus compositions, films, slurries, injectable solutions, gums, lozenges, and the like. In addition, there is a need for compositions that penetrate openings in teeth, permit visualization of such openings within teeth, and facilitate or enhance delivery of active ingredients into the teeth and surrounding tissues. Still further, there is an unmet need in the art for compositions that facilitate or enhance the delivery to the oral cavity of chemical elements and active ingredients that prevent or ameliorate disorders and diseases of teeth and gingiva. Lastly, there is an unmet need for methods of use and preparation of the aforementioned compositions.

Due to the inadequacy of mechanical removal of plaque via tooth brushing, there is much interest in chemical inhibition of plaque formation.

### SUMMARY OF THE INVENTION

Accordingly, it is an object of this invention to provide improved oral compositions containing polyphenols including aglycone flavones, flavonols, and flavanols. Such compositions are toothpastes, mouthwashes, mouth rinses, gum, candy, as well as in toothache, sore throat, and cold sore medications.

It is another object of the invention to provide improved oral compositions containing a combination of aglycone flavones, aglycone flavonols, aglycone flavanols and other relatively insoluble polyphenol ingredients.

It is another object of this invention to provide stable formulations containing soluble alkali metal polyphenol salts, e.g. flavone salts with the oxidative sodium bicarbonate and sodium carbonate peroxide combination.

The present invention relates to polyphenol containing compositions for use in the preparation of oral compositions, such as toothpastes, mouthwashes or mouth rinses, gums, and candies. In particular, it relates to oral compositions comprising antibacterial, antioxidant, anti-inflammatory polyphenol/flavonoid formulations, their preparation and use. The invention discloses oral products containing therapeutically effective concentrations of aglycone flavonoid compounds as antiplaque and anti-inflammatory agents within suitable oral vehicles or carriers (“orally acceptable”—not harmful to the patient when used in the mouth) for treating oral inflammatory disorders and inhibiting and/or killing bacteria. The amounts used of the polyphenol are “therapeutically effective,” i.e., amounts needed as part of the composition to obtain the desired result such as reducing dental caries, symptoms of gingivitis, periodontitis, inhibiting the activity of glucosyltransferase, etc. in mammals including humans. The amounts used of the polyphenol may also be “prophylactically effective” amounts meaning that the amounts may prevent the onset or occurrence of oral cavity disorders and diseases such as dental caries, symptoms of gingivitis, periodontitis, and inhibiting the activity of glucosyltransferase in mammals including humans.

The present invention, in various embodiments, provides oral compositions for preventing, treating and/or inhibiting various dental diseases, such as dental caries, gingivitis, periodontitis, etc. The oral compositions can be present in various different forms. For example, the oral compositions can be at least one of a dentifrice, paste, gel, powder, mouth rinse, mouthwash, tooth hardener, oral film, anti-calculus composition, film, slurry, injectable solution, and lozenge.

Microemulsions of relatively insoluble aglycone polyphenols are disclosed to improve the aqueous solubility within these oral hygienic products. The methods of production include both the formation of both a high temperature surfactant/polyphenol concentrate, and a nano-particulate precipitation process in the presence of a sufficient surfactant concentration.

Soluble flavone salt formulations within alkaline oral compositions containing sodium bicarbonate, sodium carbonate peroxide and a peroxide stabilizer have been experimentally determined to be effective for treating, oral inflammatory disorders and inhibiting bacterial growth.

The alkaline soluble polyphenol salt containing toothpaste compositions of the present invention can include solid inorganic peroxide which will allow the release of nascent oxygen upon brushing of the teeth with the composition such that the nascent oxygen is activated and released upon contact with the saliva in the mouth or the addition of water. Further, a water soluble or water emulsifiable coating encapsulates the peroxide ingredient.

The invention provides stable formulations containing soluble alkali metal flavone salts with the oxidative sodium bicarbonate and sodium carbonate peroxide combination.

The invention relates to a composition comprising, consisting of, or consisting essentially of:

i) a polyphenol, and

ii) an orally acceptable carrier,

wherein said polyphenol is in the form of an alkali metal salt or a concentrate.

Typical polyphenols include flavonoids and stilbenes.

Typical carriers are water (e.g. deionized), glycerin, ethanol, sorbitol, and propylene glycol. Additives are included depending on the form of the composition, e.g. toothpaste, mouth wash, and muco-adhesive vehicle.

The compositions inhibit the activity of surface bound glucosyltransferase, and inhibit or destroy microorganisms (particularly those producing glucosyltransferase) upon administration to an oral cavity.

In an advantageous embodiment, the composition does not include DMSO, and/or the composition does not include greater than 40% ethanol. Typically the polyphenol is 0.01-20 percent by weight of the composition. The composition generally is in the form of a liquid, a gel, a paste, a spray, a powder, a gum, a lozenge or a tablet. In an advantageous embodiment, the composition, e.g. toothpaste, comprises: a flavonoid, sodium bicarbonate, and a peroxide, e.g. sodium carbonate peroxide, and optionally, a polymer for stabilizing the sodium bicarbonate and/or peroxide.

The invention also relates to a method of inhibiting the activity of surface bound glucosyltransferase, and inhibiting the activity of soluble and surface-bound microorganisms responsible for dental caries comprising: administering to the oral cavity a therapeutically effective amount of a composition of the invention.

In another embodiment, the invention relates to a method of providing therapeutically effective levels of a polyphenol in an oral cavity comprising administering to the oral cavity a composition of the invention.

In a still further embodiment, the invention relates to a method of delivering a polyphenol systemically to a mammal comprising administering buccally to the oral cavity of a mammal a composition of the invention, typically at least once weekly, or advantageously once a day.

In another embodiment, the invention relates to a method for preventing or treating an oral disease or condition in a mammal comprising: administering to the oral cavity of said mammal a sustained therapeutically effective amount of a composition of the invention. Administering to the oral cavity comprises administering to one or more of a tooth, a mucosal surface, a tongue surface, a surface on complete or partial dentures, or a combination thereof. The composition is administered at least once daily. Alternatively, the composition in the form of a rinse is administered to the oral cavity for a period of about 30-60 seconds. In the form of a paste or gel, the composition is administered to the oral cavity for a period of at least 1 minute. Another embodiment of the invention is dental floss coated with a composition of the invention.

In a further embodiment, the invention relates to a method of making a polyphenol containing toothpaste or oral rinse composition comprising:

a) mixing a heat stable polyphenol compound with a heat stable nonionic surfactant [(e.g. PS80 and Polyoxyl-40-hydrogenated castor oil (Cremophor/Kolliphor RH-40)] to form a mixture,

b) heating said mixture resulting from step a) to a temperature such that said heat stable polyphenol compound is solubilized to form a concentrate,

c) cooling said concentrate resulting from step b),

d) adding the solubilized polyphenol concentrate of step c) to a toothpaste or oral rinse to form said polyphenol containing toothpaste or oral rinse composition.

In another embodiment, the invention relates to a method of making a polyphenol containing toothpaste or oral rinse composition comprising:

a) mixing a polyphenol with a toothpaste or oral rinse formulation to form a composition,

b) adding an alkali metal hydroxide to the composition of step a) to a pH level of about 10 to form an alkali metal polyphenol salt within said toothpaste or oral rinse formulation, and

c) acidifying the product of step (b) with an acidic agent to form said polyphenol containing toothpaste or oral rinse composition.

The alkali metal hydroxide is typically sodium hydroxide or potassium hydroxide or a mixture thereof, and the acidifying agent is citric acid, acetic acid, ascorbic acid, hydrochloric acid or a mixture thereof.

Further, the invention includes a multi-cavity dispensing container for delivering a polyphenol and a toothpaste comprising: a first cavity containing a toothpaste or gel, and a second cavity containing a gel or paste containing a polyphenol (e.g. flavonoid). The container prevents the interaction between the polyphenol and the toothpaste which can be alkaline) prior to the time the components contact each other on a toothbrush.

Another object of the invention is to provide orally-acceptable compositions that persist in the oral cavity for prolonged periods of time for the prevention or treatment of oral cavity disorders and diseases including those involving teeth and gingiva.

Another object of the invention is to provide orally-acceptable compositions that penetrate teeth through cracks, dentinal tubules, caries, and the cementoenamel junction so as to enhance the delivery and efficacy of prophylactic- or therapeutic active agents, and to enhance visualization of openings in teeth.

Yet another object of the invention is to provide compositions that facilitate or enhance the delivery of calcium, zinc, magnesium, and other chemical elements to teeth and surrounding soft tissue for the prevention or treatment of various dental disorders and diseases.

Further objects of the invention are to provide methods of using, and methods of making, the aforementioned compositions of the invention (as well as kits comprising such compositions).

In a representative embodiment, a method is provided for preventing or treating disorders or diseases of the oral cavity. The method involves administering a composition that comprises a solubilized polyphenol, solubilized flavonoid, solubilized curcuminoid, or combination of two or more solubilized polyphenols, two or more solubilized flavonoids, two or more solubilized curcuminoids, or combinations of solubilized flavonoids and solubilized curcuminoids. The solubilized polyphenol, solubilized flavonoid, solubilized curcuminoid, or combinations thereof persist in the oral cavity of an animal (such as, for example, a mammal including a human) longer than nonsolubilized polyphenol, nonsolubilized flavonoid, nonsolubilized curcuminoid, or combinations thereof. In representative embodiments, the solubilized flavonoid may be, for example, apigenin, luteolin, kaemferol, quercetin, myricetin, daidzein, genistein, catechins, gallocatechins, naringin, rutin, hesperitin, anthocyanidins, and combinations thereof. In other representative embodiments, the solubilized curcuminoid may be, for example, curcumin, tetrahydro curcumin, and combinations of curcuminoids. The ranges of solubilized polyphenol, solubilized flavonoid, solubilized curcuminoid, or combinations thereof, in the orally-acceptable formulations may be, for example, 0.5%-4.0%, 1.0%-2.0%, and 0.1% to 20%. The disorders or diseases of the oral cavity may be, for example, dental plaque, dental caries, periodontal disease, oral cancer, oral chemotherapy sequelae, gingivitis, herpetic lesions, cold sores, aphthous ulcers, dry mouth, toothache, wound, tooth sensitivity or pain, denture stomatitis, fungal, viral or bacterial infections, and combinations thereof. In alternative embodiments, the solubilized polyphenol, solubilized flavonoid, solubilized curcuminoid, or combinations thereof persist in the oral cavity for up to at least three, six, and twelve hours. Persistence of the solubilized polyphenol, solubilized flavonoid, solubilized curcuminoid, or combinations thereof is at least three, six, and twelve hours longer than the nonsolubilized polyphenol, nonsolubilized flavonoid, nonsolubilized curcuminoid, or combinations thereof.

In another representative embodiment, the solubilized polyphenol, solubilized flavonoid, curcuminoid, or combinations thereof penetrate a dental structure. In alternative embodiments, the dental structure may be, for example, plaque, tartar, calculus, caries, cracks, dentinal tubules, sulcus, and cementoenamel junctions.

In further embodiments, the solubilized polyphenol, solubilized flavonoid or curcuminoid may be prepared by, for example, chelation, heat solubilized concentration, alkali metal salting, and combinations thereof.

In other representative embodiments, a composition is provided for that comprises a chelated flavonoid or chelated curcuminoid, combinations thereof, and an orally-acceptable carrier. The chelated flavonoid or chelated curcuminoid comprises an alkali metal, which may be, for example, calcium, zinc, magnesium, iron, copper, and boron. In one aspect, the active flavonoid or active curcuminoid is released from the chelated flavonoid or chelated curcuminoid upon acidification of the composition. In alternative aspects, the acidification step may be, for example, reducing the pH to, for example, pH 4, pH 3, pH 2, and lower.

In an alternative embodiment, a method is provided for administering to the oral cavity of a subject (for example, a mammal such as a human) compositions comprising concentrations of solubilized polyphenols, solubilized flavonoids, and solubilized curcuminoids in the range of, for example, 0.5%-4.0%, 1.0%-2.0%, and 0.1% to 20% by weight of the composition, to achieve prevention or treatment of, for example, oral disorders and diseases including dental plaque, dental caries, periodontal disease, oral cancer, oral chemotherapy sequelae, gingivitis, herpetic lesions, cold sores, aphthous ulcers, dry mouth, toothache, wound, tooth sensitivity or pain, denture stomatitis, fungal, viral or bacterial infections, and combinations thereof. The compositions comprising solubilized polyphenols, solubilized flavonoids, and solubilized curcuminoids can be administered once or multiple times each day based upon severity of disorder or disease and as determined by one skilled in the art to achieve desired results. Following any single administration, the solubilized compositions persist in the oral cavity for at least three, six, and twelve hours longer than single administration of compositions comprising nonsolubilized polyphenol, nonsolubilized flavonoid, nonsolubilized curcuminoid, or combinations thereof. The orally-acceptable compositions comprising solubilized polyphenols, solubilized flavonoids, and solubilized curcuminoids can be administered to the oral cavity by various means well known to those skilled in the art including, but not limited to, tooth brushing.

These and other objects will become apparent to those skilled in the art upon a further reading of the specification.

### DETAILED DESCRIPTION OF THE INVENTION

The subject invention relates to compositions comprising antibacterial, antioxidant, anti-inflammatory polyphenol/flavonoid compositions, their preparation and use. It is desirable to incorporate flavones particularly apigenin, luteolin, quercitin, etc., as part of oral formulations to aid in the maintenance of proper oral hygiene. The methods associated with preparation of polyphenol/flavonoid containing compositions are useful in the preparation of oral compositions, such as toothpastes, mouthwashes or mouth rinses, gums, drinks and candies. See commonly owned U.S. patent application Ser. No. 14/215,984 hereby incorporated by reference in its entirety.

The oral compositions are useful for preventing, treating and/or inhibiting various oral inflammatory conditions, such as gingivitis, periodontitis, etc. The compositions are well suited to inhibit the buildup of microorganisms that promote gingivitis, dental caries and the development of the dental plaque structure. Plaque is a haven for oral microorganisms and continues to build up in the oral cavity until it can mineralize to form calculus (also known as tartar) as well as cause plaque associated gum disease. The microorganisms that form the biofilm can be Streptococcus mutans and anaerobes depending upon, for example, the patient age, location in the mouth, and salivary flow.

Toothpaste and oral rinses are an essential part of oral hygiene since they disrupt the biofilm, plaque and tartar to help prevent disease. Gum disease, or gingivitis, occurs when there is an abundance of bacteria and subsequent plaque present around teeth. Untreated cases can eventually lead to tooth loss, so it is important to exercise daily preventive measures that include toothpaste for the treatment and prevention of gum disease.

Gingivitis (gum inflammation) often precedes periodontitis (gum disease). However, not all gingivitis progresses to periodontitis. If left untreated, gingivitis may progress to periodontitis which can be a major cause of tooth loss in adults.

Microemulsions of relatively insoluble aglycone polyphenols are disclosed to improve their aqueous solubility within oral hygienic compositions.

The methods of making the compositions include i) the formation of a high temperature polyphenol/surfactant concentrates, ii) a nano-particulate precipitation process in the presence of a surfactant, and iii) the solubilization of relatively insoluble aglycone polyphenols/flavonoids within alkaline oral compositions and iv) combinations thereof.

The invention includes methods for increasing the solubility of poorly soluble aglycone flavonoid compounds with surfactants such as polysorbate, polyoxyl hydrogenated castor oil, etc. in formulations. As previously noted, many aglycone flavonoids and specifically apigenin are poorly soluble in aqueous solutions thus severely limiting their bioavailability for oral, pharmaceutical and nutraceutical applications.

Compositions of the Invention

The polyphenol compositions of the invention can be present in various forms. For example, the compositions can be in the form of a dentifrice (toothpaste), paste, gel, powder, liquid, mouthwash, mouth rinse, cream, lotion, tooth hardener, oral film, anti-calculus composition, film, slurry, injectable solution, gum, lozenge, tablet, candy, food or beverage. The compositions can take the form of a solution (e.g., mouthwash), a suspension, or an emulsion. These compositions include a suitable carrier for the aglycone flavonoid/polyphenol ingredients.

The compositions of this invention are compositions of polyphenols. The polyphenols include flavonoids, stilbenes, curcumins, and lignans. As used herein, “poorly soluble” or “relatively aqueous insoluble” are polyphenols or flavonoids having a solubility in water of less than 1 mg/ml, and particularly less than 0.1 mg/ml.

It has been found that improved solubility and stability of the aqueous aglycone flavonoid microemulsions can be greatly improved by adjusting the pH by the addition of pH-controlling agents, buffers, to maintain pH within a range of from 5.5 to 11.0.

Flavonoids

Flavonoids of the invention include flavones, flavonols, flavanols, proanthocyanidins, dihydroflavonols, flavones, and derivatives thereof. Exemplary are aglycone flavonoids without limitations and include apigenin, luteolin, chrysin, quercetin, hesperitin, naringin, genistein, daidzein, epigallocatechin gallate, catechin and combinations thereof, e.g. apigenin and luteolin and/or ECGC.

In another embodiment, combinations (for use in, for example, a toothpaste, gum or mouth rinse) include a flavonoid (e.g. apigenin and luteolin and/or ECGC) and a non-flavonoid polyphenol (e.g. curcumin and/or resveratrol).

The chemical structures of some commonly occurring plant flavonoids are listed in Table I.

TABLE I
CHEMICAL STRUCTURES OF SOME COMMONLY OCCURING PLANT FLAVONOIDS

| Structure | Represtative flavonoids |
|---|---|
| Flavones | R1 = H, R2 = OH: Apigenin |
| 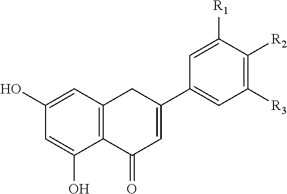 | R1 = R2 = OH: Lutcolin |
| Flavonols | R2 = OH, R1 = R3 = H: Kaempferol |
| 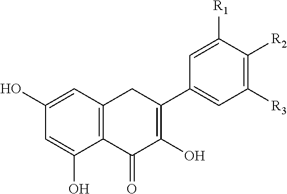 | R1 = R2 = OH, R3 = H: Quereetin R1 = R2 = R3 = OH: Myrieetin |
| Isoflavones | R1 = H: Daidzein |
| 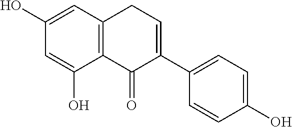 [SMILES: O=C1C(c2ccc(O)cc2)=CCc2cc(O)cc(O)c21] | R1 = OH: Genistein |
| Flavonols | R1 = R2 = OH, R3 = H: Catechins |
| 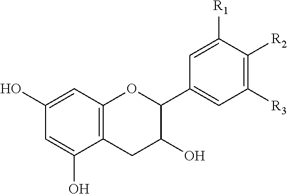 | R1 = R2 = R3 = OH: Gallocatechin |
| Flavanones | R1 = H, R2 = OH: Naringenia |
| 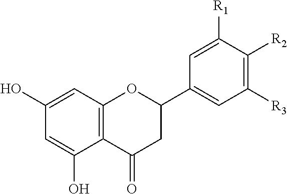 | R1 = R2 = OH: Enodicyrol R1 = OH, R2 = OCH3: Hespereum |

Apigenin is a member of the flavone structural class and is chemically known as 4′,5,7,-trihydroxyflavone. Apigenin has the following structural formula:

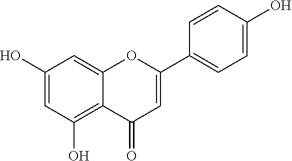 [SMILES: O=c1cc(-c2ccc(O)cc2)oc2cc(O)cc(O)c12]

Luteolin is also a member of the flavone structural class and is chemically known as 3′,4′,5,7-tetrahydroxyflavone. Luteolin has the following structural formula:

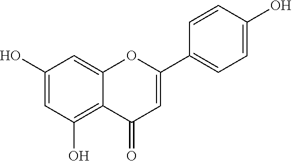 [SMILES: O=c1cc(-c2ccc(O)cc2)oc2cc(O)cc(O)c12]

Epigallocatechin gallate (EGCG) is a type of catechin that is a most abundant in tea and is a potent antioxidant that may have therapeutic application in the treatment of many disorders. EGCG has been found to effective in the treatment of Sjogrens syndrome. EGCG has the following structural formula:

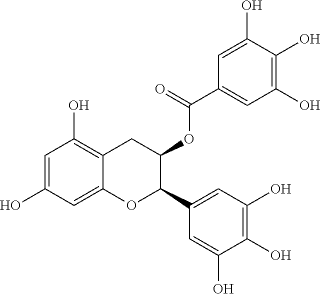 [SMILES: O=C(O[C@@H]1Cc2c(O)cc(O)cc2O[C@@H]1c1cc(O)c(O)c(O)c1)c1cc(O)c(O)c(O)c1]

The aglycone flavonoid is present in the formulations of the invention in amounts to inhibit the activity of soluble or surface-bound bacterial microorganisms so as to prevent or to treat dental caries, dental plaque formation, gingivitis, periodontitis, candidiasis, dental stomatitis, and fungal infections.

An effective amount of polyphenol, e.g. aglycone flavonoids, present in the oral compositions of the invention are greater than 0.01 wt. %, greater than or 0.1 wt. %. Typically, the aglycone flavonoid is present in an amount that is between 0.1 to about 20 wt. %, or from about 0.3 to about 20 wt. %, or from 7 wt. % to about 20 wt. %.

Curcumin is a diarylheptanoid. It is the principal curcuminoid of turmeric, which is a member of the ginger family (Zingiberaceae). Turmeric's other two curcuminoids are desmethoxycurcumin and bis-desmethoxycurcumin. The curcuminoids are natural phenols that are responsible for the yellow color of turmeric. Curcumin can exist in several tautomeric forms, including a 1,3-diketo form and two equivalent enol forms. The enol form is more energetically stable in the solid phase and in solution, and is reportedly unstable and degrades quickly in alkaline preparations. Curcuminoids as used herein also comprise the hydrogenated derivatives of curcuminiods such as tetrahydro-curcumin.

Carriers and Additives

The particular choice of carrier will depend, at least in part, upon the desired form of the oral composition: for example a toothpaste or gel, a powder, a solution (e.g., mouthwash or mouth rinse), a suspension, an emulsion, a lozenge, a mucoadhesive vehicle, a beverage a tablet, a capsule or a gum. Conventional ingredients that can be used to form the carriers listed above are well known to the skilled artisan. Any suitable orally acceptable vehicle can be used, such as those described in U.S. Pat. No. 4,894,220 hereby incorporated by reference in its entirety.

Preferably, such carrier materials are selected for compatibility and stability with all of the constituents of the formulation including the active ingredient(s), such as aglycone flavonoid(s) and the optional one or more oral care active agent compounds selected for the oral composition. Further, as described previously above, the carrier ingredient can also serve as a bioavailability-enhancing agent, either as an efficacy-enhancing agent or a solubilizing agent for the active ingredients.

As recognized by one of skill in the art, the oral compositions optionally include other materials in addition to those components previously described, including for example without limitation, a cariostatic agent, a humectant, an abrasive agent, a gelling agent, a flavoring agent, a desensitizing agent, an anti-calculus agent, a whitening agent, a surfactant, a binding agent, a preservative, a buffering agent, an opacifying agent, a coloring agent, and combinations thereof. It is understood that while general attributes of each of the above categories of materials may differ, there may be some common attributes and any given material can serve multiple purposes within two or more of such categories of materials.

Water is typically an element of the oral compositions. Water employed in the preparation of commercially suitable toothpastes should advantageously be deionized and free of organic impurities. The amounts of water include the free water which is added plus that which is introduced with other materials. In certain embodiments, the oral compositions are anhydrous; e.g., stannous fluoride and calcium sodium phosphosilicate formulations. In another embodiment, the amount of water is less than 5 wt. % (e.g., PeroxiCare® type formulation).

The oral composition can be a liquid, such as a mouthwash or mouth rinse which typically contains an aqueous non-toxic lower aliphatic alcohol, advantageously having about 2-30 wt. % by weight of a non-toxic alcohol, such as ethanol, n-propanol, or isopropanol, with water, and often about 5-35 percent of humectant.

Cariostatic agents (non-flavonoid cariostatic agents) can be provided in each form of the oral composition. Fluoride in various forms is the most popular active ingredient in toothpaste to prevent cavities. The additional fluoride in toothpaste has beneficial effects on the formation of dental enamel and bones. Suitable cariostatic agents include sodium fluoride, stannous fluoride, aminefluoride, sodium monofluorophosphate, sodium trimeta-phosphate, triclosan, casein, or combinations thereof. If desired, the cariostatic agent can be present in an amount between about 0.01 to about 2 weight percent, more typically between about 0.02 to about 1 weight percent.

Humectants can also be employed in the oral compositions, particularly toothpastes and gels and oral rinses. These agents are used to give toothpaste texture, prevent drying out by retaining moisture and prevent hardening of the paste on exposure to air. Suitable humectants include, glycerin, propylene glycol, polyethylene glycol, xylitol, sorbitol, maltitol, lactitol, or the like. The humectant can also be used as the bulk carrier, in which case it can be present in an amount of about 5 to about 90 weight percent, more typically about 10 to about 60 weight percent.

Abrasive agents are typically employed in dentifrice compositions. Abrasives constitute at least 50 wt. % of typical toothpaste. These insoluble particles help remove plaque from the teeth. The removal of plaque and calculus helps minimize cavities and periodontal disease. Suitable abrasive agents include silica gel, zirconosilicate, silicic anhydride, aluminosilicate, calcium carbonate, calcium pyrophosphate, aluminum oxide, aluminum hydroxide, calcium hydrogen phosphate dihydrate or anhydride, aluminum silicate, insoluble sodium metaphosphate, magnesium carbonate, calcium sulfate, and combinations thereof. Sodium bicarbonate is a particularly effective abrasive agent that also provides a mild teeth-whitening action. It neutralizes acidic saliva, thus maintaining an alkaline environment in the mouth, even hours after brushing. An alkaline environment is not favorable for and hampers the formation of dental plaque. It is a natural teeth whitener and hence effective to remove stains. It is an effective teeth-cleaning agent and due to its abrasive action it can clear off those brown and yellow stains. While brushing, baking soda infiltrates the tooth's enamel, which helps to reduce the appearance of the stains that are on the surface of the teeth. Abrasives can generally be employed in effective amounts of between about 20 to about 90 weight percent, more typically about 20 to about 60 weight percent.

Gelling agents or thickeners can be used in the various compositions. Suitable gelling agents include carrageenan, sodium carboxymethyl cellulose, alkali metal alginates such as sodium alginate, gums, polyvinyl alcohol, and veegum or the like. Typically, the gelling agents are employed in amount of about 0.3 to about 5 weight percent.

Flavorants in toothpaste comes in a variety of colors and flavors intended to encourage use of the product. Three most common flavorants are peppermint, spearmint, and wintergreen. The respective oils, e.g. peppermint oil, provide these flavors. More exotic flavors include anise, apricot, bubblegum, cinnamon, fennel, lavender, ginger, vanilla, lemon, orange, and pine. Unflavored toothpastes exist.

Desensitizing agents can be introduced in some of the oral composition to alleviate sensitivity of individuals whose teeth are sensitive to thermal shock, chemicals, etc. Suitable desensitizing agents include potassium nitrate, potassium citrate, potassium chloride, potassium tartrate, potassium bicarbonate, potassium oxalate, and strontium salts. Desensitizing agents can be present, either individually or collectively, in an amount of about 0.1 to about 5 weight percent, more typically about 0.1 to about 3 weight percent.

Anti-calculus agents can be introduced to the oral composition to treat tartar formation. Suitable anti-calculus agents include alkali-metal pyrophosphates, hypophosphite-containing polymers, organic phosphonates, phosphocitrates, zinc salts and combinations thereof. Anti-calculus agents can be present, either individually or collectively, in an amount of about 0.1 to about 5 weight percent, more typically about 0.1 to about 3 weight percent.

Whitening agents can be employed in some forms of the oral composition. Some of these toothpastes contain peroxide, the same ingredient found in tooth bleaching gels. Suitable whitening agents include sodium carbonate peroxide, calcium peroxide, sodium tripolyphosphate and hydrogen peroxide. Whitening agents can be employed in amounts of about 0.5 to about 5 weight percent.

Surfactants can also be employed in the various oral compositions. The purpose of these agents is to facilitate the distribution of the paste in the mouth by lowering the surface tension and helping to loosen plaque and other debris from the tooth surface. They also contribute to the foaming action of toothpastes. Fluorides work better in combination with detergents, which help the remineralization process of tooth enamel. Any of a variety of types of surfactants can be utilized, including anionic, nonionic, cationic and zwitterionic or amphoteric surfactants, or combinations thereof. Exemplary anionic surfactants include, without limitation, sodium lauryl sulfate, sodium lauryl sarcosinate, a-olefin sulfate, tabulate, lauryl monoglyceride sulfate, lauryl monoglyceride sulfonate, and combinations thereof. Exemplary nonionic surfactants include, without limitation, TWEEN, lauroyl diethanol amide, stearyl monoglyceride, sucrose fatty acid esters, lactose fatty acid esters, lactitol fatty acid esters, maltitol fatty acid esters, polyoxyethylene sorbitan monostearate, and combinations thereof. Exemplary ampholytic surfactants include, without limitation, betain and amino acid type surfactants. Surfactants can be present in amount of about 0.5 to about 15 weight percent, more typically about 0.5 to about 10 weight percent.

Binding agents maintain the consistency of toothpaste, tablet or lozenges. It binds all the ingredients in the formulation together. Hydrocolloids, such as alginate or xanthan, are often used as binding agents. Other binding agents include sodium carboxymethyl-cellulose, gum arabic as well as synthetic polymers such as polyacrylates and carboxyvinyl polymers. Binders can be present in amounts of about 0.5 to about 50 weight percent depending on the form of the oral composition.

Preservatives play an important role in keeping the oral compositions free from microorganisms. Sodium benzoate is a commonly used preservation agent that prevents the buildup of microorganisms in oral products and also functions to provide a degree of cariostatic activity. Other commonly used preservatives in oral compositions include, methyl paraben, and ethyl paraben.

Buffering agents useful in the present compositions are those that are capable of maintaining the desired pH thereby promoting its stability and desired properties. The pHs of oral compositions are generally in the range of about 4.5 to about 11, or about 6.5 to about 9.0. The pH can be adjusted with the addition of acidic ingredients such as citric acid or benzoic acid or alkaline ingredients such as sodium or potassium hydroxide and buffered to maintain pH with salts such as sodium citrate, benzoate, carbonate, or bicarbonate, disodium hydrogen phosphate, sodium dihydrogen phosphate, etc.

Opacifying agents can also be added to various oral compositions of the present invention. Titanium dioxide is a white powder that adds opacity to the compositions. Titanium dioxide can be present in an amount of about 0.25 to about 5 weight percent.

Coloring agents provide toothpaste with pleasing colors. Artificial dyes are used to make red, green, and blue toothpastes. Coloring agents can be present in an amount of about 0.01 to about 5 weight percent.

Other ingredients such as minerals, vitamins, herbs, CoQ10, propolis, echinacea etc., are often added in the formulation of toothpaste, to make it more effective in controlling bad breath and plaque formation. Vitamins include Vitamins C (L-ascorbic acid) and D, thiamine, riboflavin, calcium pantothenate, niacin, folic acid, nicotinamide, pyridoxine, cyanocobalamin, para-aminobenzoic acid, bioflavonoids, and mixtures thereof.

The term “penetration” as used herein means, but is not limited to, the process by which an active substance or ingredient (such as, a polyphenol or flavonoid) enters or is found within a dental structure.

The terms “leeching” and “leech(es)” as used herein mean, but are not limited to, the process by which an active substance or ingredient (such as, a polyphenol or flavonoid) is released from the tooth surface or internal portion of tooth into saliva over time.

The term “persistence” as used herein means, but is not limited to, the amount of time an active substance or ingredient (such as, a polyphenol or flavonoid) is present in the target tissue area.

The term “carious tooth” as used herein means, but is not limited to, a tooth with an unrepaired dental decay (cavity) in it, which may be due to activities of bacteria. Symptoms may include pain and difficulty with eating. Complications may include inflammation of the tissue around the tooth, tooth loss, and infection or abscess formation.

The term “virgin tooth” as used herein means, but is not limited to, a tooth without cavity (repaired or unrepaired), obvious cracks, or broken portions.

The term “sulcus” as used herein is the area around the tooth where the gum attaches to the tooth.

The term “muco-adhesive” as used herein is any compound that adheres to the tissues in the mouth.

The term “chelate” as used herein means, but is not limited to, a chemical compound in the form of a heterocyclic ring, containing a metal ion attached by coordinate bonds to at least two nonmetal ions. The term “chelation” as used herein means, but is not limited to, the particular way that ions and molecules bind metal ions. Chelation involves the formation or presence of two or more separate coordinate bonds between a polydentate (multiple bonded) ligand and a single central atom. Ligands are typically organic compounds, and are called chelants, chelators, chelating agents, complexing or complex-forming agents, or sequestering agents. Some chelates are reversible in acidic conditions; when exposed to pHs below 4, they break apart and release the chelated metal and the chelating compound. Polyphenol chelates have demonstrated this characteristic, starting to break apart at a pH of 4 and more actively breaking apart at pHs of 3, 2, or even more acidic pHs. One skilled in the art would understand that the chelated polyphenols, chelated flavonoids, chelated curcuminoids, and combinations thereof, may be used in other applications besides the prevention or treatment of diseases and disorders of the oral cavity. Further, one skilled in the art would understand that the chelated polyphenols, chelated flavonoids, chelated curcuminoids, and combinations thereof may be prepared as various formulations for alternative applications and that compositions comprising chelated polyphenols, chelated flavonoids, chelated curcuminoids, and combinations thereof may be used, for example, as pharmaceuticals, medical devices, nutraceuticals, and cosmeceuticals.

Polyphenol/Surfactant Concentrate Formulations

The polyphenol/surfactant “concentrates”, see commonly owned U.S. Pat. No. 8,637,569 hereby incorporated by reference in its entirety, can be utilized to form a variety of oral compositions including toothpaste or gel, a powder, a solution (e.g., mouthwash or mouth rinse), a suspension, an emulsion, a lozenge, a tablet or a gum.

Polyphenol Microemulsion Formulations

The polyphenol/surfactant microemulsions, see commonly owned U.S. Patent Application 2012/0213842 hereby incorporated by reference in its entirety, can be utilized to form a variety of oral compositions including toothpaste or gel, a powder, a solution (e.g., mouthwash or mouth rinse), a suspension, an emulsion, a lozenge, a tablet or a gum.

Polyphenol Salt Formulations

Advantageous embodiments of the invention are stable formulations containing aqueous soluble alkali metal polyphenol, e.g. flavone salts. The soluble alkali flavone ingredients, advantageously the sodium salt of apigenin, within the compositions, e.g. dentifrice compositions, comprise 0.01 to 20 wt. %, 0.1 wt. % to 20 wt. %, or from 0.3 wt. % to 20 wt. %.

The dentifrice compositions of the present invention including alkali metal salts of a polyphenol such as a flavone, typically contain a limited water concentration, i.e. from 1 wt. % to 5 wt. % and more advantageously, from 2 wt. % to 3 wt. %.

Particularly advantageous embodiments of the invention are stable formulations containing soluble alkali metal polyphenol salts, e.g. flavone salts with the oxidative sodium bicarbonate and peroxide, e.g. sodium carbonate peroxide combination.

Suitable peroxides in the composition include encapsulated solid inorganic peroxides advantageously alkali metal carbonate peroxides such as sodium carbonate peroxide which will allow the release of nascent oxygen upon brushing of the teeth with the composition. The nascent oxygen is generated and released upon formulation contact with the saliva in the mouth or the addition of water. Sodium carbonate peroxide concentrations comprise from about 2 wt. % to about 10 wt. % and advantageously from 1 wt. % to about 10 wt. %.

The bicarbonate salt ingredients, advantageously sodium bicarbonate, of the dentifrice compositions comprise about 10 wt. % to about 60 wt. % of the composition and advantageously from about 20 wt. % to about 50 wt. %.

The stabilizing material is included in the composition of the invention in an amount effective so as to inhibit breakdown of the peroxide, the soluble aglycone flavonoid salt and/or sodium bicarbonate in the composition during storage in a closed container, but at a concentration sufficient so as to allow release of nascent oxygen from the peroxide when the composition is in contacted with saliva during brushing of teeth. Suitable stabilizing ingredients include polymer compositions include PEG and PEG/PPG copolymers. Ascorbic acid may be added to the formulation to stabilize alkali metal aglycone flavonoid salts. The stabilizing ingredients are included in the composition in an amount of from 1 wt. % to about 20 wt. % and advantageously from about 2 wt. % to about 10 wt. %.

Several polyphenol and flavonoid ingredients are unstable in alkaline oral compositions over time. Alkaline stability concerns can be addressed by a two cavity dispensing system such that the alkaline oral compositions (in first cavity) and the polyphenol ingredients (in second cavity) are dispensed on a toothbrush just prior to brushing. See U.S. Pat. Nos. 5,814,303, 6,230,935, 6,547,101 and 5,020,694 each of which is hereby incorporated by reference in its entirety.

In another embodiment of the invention, a solid alkali metal flavonoid salt, such as a salt of a flavone (e.g. apigenin or luteolin) is prepared. When this solid (which can be in powder form) is added to water (or saliva) the salt dissolves. This solid salt can be used in a variety of products that do not contain water including dietary supplements and foods. See Example 18.

Representative compositions comprise a polyphenol and an orally acceptable carrier. The polyphenol may be, for example, in the form of an alkali metal salt or a concentrate. Solid alkali salt compositions (such as, powders) of polyphenols may be used, for example, in toothpastes, non-aqueous compositions such as soft gels, two-piece capsules or tablets, nutraceutical supplements, and other over-the-counter compositions. The compositions of the present invention may also be used, for example, as pharmaceuticals, medical devices, and cosmeceuticals. The composition inhibits accumulation of microorganisms upon administration to an oral cavity. A representative composition does not include DMSO. Another representative composition does not include greater that 40% ethanol. A polyphenol may be, for example, a flavonoid, and the flavonoid may be, for example, apigenin, lutcolin, kaemferol, quercetin, myricetin, daidzein, genistein, catechins, gallocatechins, naringin, rutin, hesperitin, anthocyanidins, and combinations of two or more of the foregoing. In addition, a polyphenol may be, for example, resveratrol or curcumin, and a polyphenol may be greater than 0.01 percent by weight of the composition. A representative polyphenol may be, for example, 0.1-20 percent by weight of the composition. A representative composition may have, for example, a pH of 5.5-8, and a representative polyphenol may be in the form of a concentrate. A composition may have a pH greater than 8, a pH greater than 10.5, a pH greater than 10, and a polyphenol may be in the form of an alkali metal salt. A representative composition may be, for example, in the form of a liquid, a gel, a paste, a spray, a powder, a gum, a lozenge or a tablet. Representative compositions may further comprise an additive such as a fluoride compound, cariostatic agent, anti-bacterial agent, anti-tartar agent, anti-inflammatory agent, and a combination of two or more thereof. Representative compositions may further comprise a compound such as a humectant, abrasive agent, gelling agent, deodorizer, whitening agent, surfactant, binding agent, preservative, coloring agent, buffering agent, stain remover, mineral, vitamin, herb, CoQ10, xylitol, and a combinations of two or more thereof. An alternative composition may further comprise ascorbic acid. Representative compositions may be in the form of, for example, a paste or gel and polyphenol may be, for example, 0.1-20 percent by weight of the composition. A composition may be in the form of a rinse or spray and a polyphenol may be, for example, 0.1-20 percent by weight of the composition. Other compositions may be in the form of, for example, a gum comprising a polyphenol dosage of at least 0.2 mg/stick of gum. An alternative composition may comprise an alkali metal polyphenol salt, sodium bicarbonate, and a peroxide. The composition may be in the form of, for example, a toothpaste or gel. Peroxide may be, for example, sodium carbonate peroxide. A composition may further comprise a polymer for stabilizing the sodium bicarbonate and/or peroxide.

A representative method comprises inhibiting the activity of soluble and surface-bound microorganisms responsible for dental caries. The methods comprises administering to the oral cavity of a mammal a therapeutically effective amount of a composition as set forth above. Another representative method provides therapeutically effective sustained levels of a polyphenol in an oral cavity of a mammal, and comprises administering to the oral cavity a composition as set forth above. Yet another representative method provides a method of delivering a polyphenol systemically to a mammal, and comprises administering buccally to the oral cavity of a mammal a composition as set forth above. A method is provided for treating an oral disease or condition in a mammal comprising administering to the oral cavity a therapeutically effective amount of a composition as set forth above. An oral disease or condition may be, for example, dental plaque, dental caries, periodontal disease, oral cancer, oral chemotherapy sequelae, gingivitis, herpetic lesions, cold sore, aphthous ulcer, dry mouth, toothache, wound, tooth sensitivity, denture stomatitis, fungal, viral or bacterial infection. Administering to the oral cavity may include, for example, administering to one or more of a tooth, a mucosal surface, a tongue surface, a surface on complete or partial dentures, and a combination of two or more thereof. A composition may be administered at least once daily. A composition may be administered to the oral cavity for a period of about 30-60 seconds, and a may be in the form of a rinse. A composition may be administered to the oral cavity for a period of at least 1 minute and a composition may be in the form of a paste or gel.

Another representative method is provided for making a polyphenol containing toothpaste or oral rinse composition comprising mixing a heat stable polyphenol compound with a heat stable nonionic surfactant to form a mixture, heating the mixture to a temperature such that the heat stable polyphenol compound is solubilized to form a concentrate, cooling the concentrate, and adding the solubilized polyphenol concentrate to a toothpaste or oral rinse to form the polyphenol containing toothpaste or oral rinse composition. The composition may be, for example, in the form of a toothpaste. The polyphenol may be, for example, a flavonoid. The nonionic surfactant may be, for example, a polysorbate. In a representative embodiment, the foregoing mixture is heated to greater than 100° C. The heat stable solubilizing compound may be, for example, a polysorbate and the flavonoid may be, for example, apigenin or luteolin.

Another representative method is provided for making a polyphenol containing toothpaste or oral rinse composition comprising mixing a polyphenol with a toothpaste or oral rinse formulation to form a composition, adding an alkali metal hydroxide to the composition to a pH level of about 10 to form an alkali metal polyphenol salt within the toothpaste or oral rinse formulation, and acidifying the product with an acidic agent to form the polyphenol containing toothpaste or oral rinse composition. The polyphenol may be, for example, a flavonoid. The heat stable solubilizing compound may be, for example, a polysorbate and the flavonoid may be, for example, apigenin or luteolin. The flavonoid may be, for example, kaemferol, quercetin, myricetin, daidzein, genistein, catechins, gallocatechins, naringin, rutin, hesperitin, anthocyanidins, and combinations of two or more thereof. The alkali metal hydroxide may be, for example, sodium hydroxide or potassium hydroxide or a mixture thereof. The acidifying agent may be, for example, citric acid, acetic acid, ascorbic acid, hydrochloric acid or a mixture thereof.

Methods of Preparing Formulations of the Invention

The Formation of Polyphenol/Surfactant Concentrate Formulations

Heating relatively aqueous insoluble polyphenol compounds to temperatures approaching their melting points with a heat stable nonionic surfactants to elevated temperatures (typically >100° C.), not exceeding the boiling point or decomposition point of either the active agent (e.g. polyphenol) or the heat stable solubilizing agent (e.g. surfactant), and then cooling said mixture, results in a “concentrate.” See U.S. Pat. No. 8,637,569.

This process can enhance dissolution, and achieve a significantly higher concentration of the polyphenolic compound in solution with the surfactant. Furthermore, the resulting solution “concentrate” is not supersaturated; such that said polyphenol/surfactant concentrate can then be used to subsequently prepare desired formulations. The molar ratio of active agent (polyphenol) to solubilizing agent (surfactant) is typically 1:2 to 1:5, and at times much greater, e.g. 1:2 to 1:20 depending on the active agent/surfactant combination. Upon cooling to room temperature, the concentrates are not supersaturated solutions even though the concentrations of the compounds are greater than their saturation concentration at ambient conditions-room temperature (temperature below that necessary to overcome the intermolecular self-association forces). The concentrate is stable and the compounds (or active agents) stay in solution at ambient temperatures for periods of time (weeks, months, advantageously 1 or 2 years) sufficient for making formulations from the concentrates.

Apigenin/Polysorbate 80 (PS80) formulations can be made as follows:
Apigenin powder and PS80 are mixed in the ratio from about 5 to 10 wt. % of apigenin to 95 to 90 wt. % PS80. This mixture is thoroughly stirred to form a paste-like blend.
The mixture is then slowly heated to relatively high temperatures to temperatures approaching 300 degree C.
A dark brown transparent liquid results such that all the solid apigenin is solubilized in the PS80 mixture.
Upon cooling to ambient temperatures, a clear viscous brown liquid results.
Based on a 5.0 wt. % concentration of apigenin in the PS80 solvent, a stable apigenin concentrate containing is 50 mg/ml is formed
It was unanticipated that high temperature levels were necessary to cause the high solubility level of apigenin and other relatively water insoluble aglycone flavonoids.

The invention includes the use of heat stable surfactants such as the polysorbate and Polyoxyl-40-hydrogenated castor oil (Cremophor/Kolliphor RH-40) surfactants to achieve elevated soluble concentrates of the other aglycone flavonoids including the flavones apigenin and luteolin, the flavonol quercetin, the flavanone hesperitin and the polyphenols resveratrol and curcumin.

The polyphenol/surfactant concentrates can be added to a variety of carriers and additives to form a toothpaste or gel, a powder, a solution (e.g., mouthwash or mouth rinse), a suspension, an emulsion, a lozenge, a tablet or a gum.

Heat Solubilization of Curcumin in Kolliphor RH-40 (Formerly Cremophor)

Step 1. A thoroughly mixed 200 g curcumin with 800 g Kolliphor RH-40 suspension (20% curcumin) was placed in a 2 L round bottom flask equipped with overhead stirring, thermometer and nitrogen inlet.

Step 2. The mixture was heated over a preheated mantle for about 15 min with stirring to 100-110° C. under nitrogen sparge to remove any moisture in the mixture.

Step 3. Under a nitrogen blanket, the mixture was heated rapidly to 150° C. to 175° C. (about 15 min). The curcumin started dissolving around 145° C., and at around 175° C. a clear deep red orange solution was obtained.

Step 4. The mixture was cooled to ambient temperature.

This method produces a 20% curcumin heat solubilized in RH-40 surfactant, forming a polyphenol concentrate.

Heat Solubilization of 10% Curcumin and 4% Apigenin in Kolliphor RH-40 (Formerly Cremophor)

Step 1. A thoroughly mixed 100 g curcumin with 900 g Kolliphor RH-40 suspension (10% curcumin) was placed in a 2 L round bottom flask equipped with overhead stirring, thermometer and nitrogen inlet.

Step 2. The mixture was heated on a preheated mantle for about 15 min with stirring to 100-110° C. under nitrogen sparge to remove any moisture in the mixture.

Step 3. Under a nitrogen blanket, the mixture was heated rapidly to 150° C. to 175° C. (about 15 min). The curcumin started dissolving around 145° C., and at around 175° C. a clear deep red orange solution was obtained.

Step 4. Using the solution from Step 3, add 40 g apigenin powder to the mixture

Step 5. The mixture above was continued to heat up to 182° C.

Step 6. The mixture was cooled to ambient temperature

A unique and clear solution of 4% apigenin/10% curcumin in RH-40 was obtained. In this process, more than 5% apigenin could be solubilized in RH-40 as curcumin is probably acting as a co-solubilizer.

Heat Solubilization of 10% Curcumin and 4% Apigenin in Polysorbate 80

Step 1. A thoroughly mixed 100 g curcumin with 900 g PS 80 suspension (20% curcumin) was placed in a 2 L round bottom flask equipped with overhead stirring, thermometer and nitrogen inlet.

Step 2. The mixture was heated on a preheated mantle for about 15 min with stirring to 100-110° C. under nitrogen sparge to remove any moisture in the mixture.

Step 3. Under a nitrogen blanket, the mixture was heated rapidly to 150° C. to 175° C. (about 15 min). The curcumin started dissolving around 145° C., and at around 175° C. a clear deep red orange solution was obtained.

Step 4. Using the solution from Step 3, add 40 g apigenin powder to the mixture

Step 5. The mixture above was continued to heat up to 185° C.

Step 6. Cool to ambient temperature.

A unique and clear solution of 4% apigenin/10% curcumin in PS 80 was obtained. In this process, more than 5% apigenin could be solubilized in PS 80 as curcumin is probably acting as a co-solubilizer.

Solubilization of Tetrahydro-Curcumin (THC) (White Curcumin) in Kolliphor RH-40

The process and profile of solubilization of white curcumin was identical with the same behavior except for the color which was very light yellow.

Step 1. A thoroughly mixed 200 g white curcumin (THC) with 800 g Kolliphor RH-40 suspension (20% curcumin) was placed in a 2 L round bottom flask equipped with overhead stirring, thermometer and nitrogen inlet.

Step 2. The mixture was heated on a preheated mantle for about 15 min with stirring to 100-110° C. under nitrogen sparge to remove any moisture in the mixture.

Step 3. Under a nitrogen blanket, the mixture was heated rapidly to 150° C. to 175° C. (about 15 min). The white curcumin started dissolving around 135° C., and at around 170° C. a clear light yellow solution was obtained.

Solubilization of Calcium-Curcumin Chelate in Kolliphor RH-40 (Formerly Cremophor)

The observed solubility of calcium-curcumin chelate was much lower than curcumin due to the inorganic complex nature of this compound. At an equivalent concentration of 20% curcumin, the chelate precipitates out. About 5% solution was stable.

Step 1. A thoroughly mixed 5 g calcium curcumin chelate with 95 g Kolliphor RH-40 suspension (5% chelate) was placed in a 250 mL round bottom flask equipped with overhead stirring, thermometer and nitrogen inlet.

Step 2. The mixture was heated on a preheated mantle for about 15 min with stirring to 100-110° C. under nitrogen sparge to remove any moisture in the mixture.

Step 3. Under a nitrogen blanket, the mixture was heated rapidly to 150° C. to 175° C. about 15 min). The chelate started dissolving around 145° C., and at around 175° C. a clear deep red solution was obtained.

Step 4. Cool solution to ambient temperature.

Synthesis of Calcium-Epigallocatechin Gallate Chelate (Ca-EGCG Chelate)

Step 1. 27.5 g (0.06 mol) of Epigallocatechin gallate (EGCG) was dissolved in 300 mL distilled water and stirred at RT under nitrogen blanket.

Step 2. 12 g of 50% NaOH solution (6 g NaOH, 0.15 mol) was slowly added to the EGCG solution and stirred under nitrogen at RT for 15 min to obtain light purple solution.

Step 3. An aqueous solution of Calcium Chloride (6.75 g, 0.06 mol, in 20 mL water) was added to above mixture, and stirred for half hour. An off white precipitate was formed, that was filtered and dried to get off white solids (25 g).

Synthesis of Calcium-Apigenin Chelate

Apigenin is quite insoluble in alcohols.

Step 1. 4 g of Apigenin (7.4 mmol) was solubilized in 600 mL of acetone under reflux conditions under nitrogen blanket.

Step 2. The solution was cooled to RT.

Step 3. 0.41 g (3.7 mmol) Calcium Chloride was added to above solution and stirred under nitrogen for 30 minutes.

Step 4. pH of the above solution was adjusted to 8.5-9.0 by drop wise addition of Ammonia/Methanol solution.

Step 5. A yellow-brown precipitate was formed that was separated by centrifugation. The precipitate was washed with water (to remove ammonium chloride) by washing with methanol. The solids were dried under vacuum to obtain dry chelate (2.5 g)

The Formation of a Flavonoid/Polyphenol Microemulsion Formulations

To prepare microemulsions of the subject invention, the teachings of commonly owned U.S. Patent Application 2012/0213842 is incorporated by reference in its entirety.

The teachings of this inventive method are applicable to preparing microemulsions of poorly soluble flavonoids/polyphenols having solubility in water less than 1 mg/ml, and particularly less than 0.1 mg/ml.

The “nano-particulate” is well suited for the addition of relatively aqueous insoluble flavonoids/polyphenols with aqueous oral compositions including mouth rinses and mouthwashes.

In one embodiment, a flavonoid/polyphenol microemulsion can be made as follows:
The mixing of a flavonoid/polyphenol with an alkali metal component (e.g., alkali metal hydroxide(s) and/or alkaline metal salt(s)) to form an alkali metal flavonoid/polyphenol salt within an aqueous solution.
Adding a surfactant in the ratio from about 5 to 20 wt. % of the flavonoid/polyphenol to 95 to 80 wt. % of a surfactant; preferable a nonionic surfactant.
This mixture is thoroughly stirred to form a uniform clear solution.
Adjusting (e.g., acidifying) the alkali metal flavonoid/polyphenol salt with an agent (e.g., an acidic agent such as acetic acid and/or hydrochloric acid) to a pH level required to form a clear and stable microemulsion.

Stable microemulsions with apigenin and the nonionic surfactants, Polysorbate 80 and Cremophor/Kolliphor RH-40, were achieved provided a pH level of 8.0 to 8.5 was maintained. For luteolin, it as determined that a pH of 7 was required to maintain a stable aqueous microemulsion; and for resveratrol, a pH of >4.5 to 7.5.

The solubilizing agent(s) (surfactant(s)) can be present in various amounts in the oral composition, such as an amount sufficient to dissolve the mixture of flavonoids/polyphenols and to prevent precipitation thereof upon dilution with saliva. The solubilizing agent(s) can also be present in an amount effective to increase the uptake of the antibacterial agent and the mixture of flavonoids/polyphenols by dental tissue. The solubilizing agent(s) are advantageously present at about 0.01 wt. % to 10% wt. %; and most advantageously, between 0.05 wt. % to 2 wt. %.

Generally toothpaste can be said to have pHs ranging between 5.5 and 11. For example fluoride will form fluoric acid and lower the pH, while baking soda or similar will increase the pH. However, many toothpaste formulations are mildly alkaline with pH ranging from 7-10 depending on its additives. The alkaline pH of toothpaste helps neutralize the plaque acids that cause tooth decay.

It was discovered that improved solubility and stability of the aqueous aglycone flavonoid (e.g., flavone and flavonol) microemulsions can be greatly improved by adjusting the pH by the addition of pH-controlling agents from about 5.5 to 11, or from 6.5 to 9.0.

In the oral cavity, bacterial metabolism releases organic acids that attack the dental enamel. Brushing of the teeth with an alkaline toothpaste will neutralize organic acids and also serves as a cleansing agent. Thus, teeth stay cleaner and avoid more damage with toothpaste. In general, the ratio of the suspended and dispersed micro-particulate form of apigenin to the dissolved alkali salt form within the vehicle is increased as the pH level of the formulation is reduced from the slightly basic (pH of approximately 8 (e.g., pH of 7 to 9) to the moderately acidic (pH of approximately 4 (e.g., pH of 3.5 to 5)).

The degree of acidity and alkalinity (pH) can have a dramatic impact on the color of selected aglycone flavonoids and can provide a qualitative colorimetric method for the determination of the presence of aglycone flavonoids. For example, the addition of alkali metal hydroxides such as sodium hydroxide to slightly acidic solutions of many aglycone flavonoids result in the formation of colored aglycone flavonoid salts. Examples of color changes due to the formation of alkali metal salts includes the yellow colored flavone and deep red/orange catechin salts.

(c) the Formation of Soluble Polyphenol Salt Formulations

As shown in the Examples below, flavone salt formulations of the invention can be formed by adding a flavone to a composition having a preexisting high pH, or alternatively mixing a flavone into a composition having a lower pH and then increasing the pH of the composition by adding an alkali metal hydroxide such as sodium hydroxide to the composition to 7.5 to 11.

It was experimentally determined that the toothpaste formulations containing both sodium bicarbonate and sodium carbonate peroxide together with stabilizing polymer formulations had pHs of ˜10-10.5. When aglycone flavone (i.e., apigenin and luteolin) powders were added to dentifrices including sodium bicarbonate, sodium bicarbonate peroxide and a peroxide stabilizer, it was also determined that soluble flavone sodium salts were formed that were stable in these highly oxidative peroxide compositions. The presence of stable soluble alkali metal flavone salts with highly anti-oxidant and anti-inflammatory properties in an emulsion formulation was totally unanticipated since aglycone flavones are prone to oxidative decomposition.

Uses of the Compositions of the Invention

The present invention provides oral compositions for treating or preventing dental diseases or conditions including dental plaque, dental caries, periodontal disease, oral cancer, chemotherapy and radiation sequelae, mucositis, gingivitis, herpetic lesion, cold sore, aphthous ulcer, toothache, wound, tooth sensitivity, denture stomatitis, fungal, viral or bacterial infection, and various oral inflammatory conditions.

The oral compositions of the present invention are also well suited to inhibit the accumulation of microorganisms which promote dental caries, gingivitis, candidiasis, denture stomatitis, or formation of dental plaques. Treating mammals by using the oral compositions of the present invention slows or stops the accumulation of microorganisms, such as Streptococci mutans.

Such oral preparations are typically applied by contacting natural or artificial teeth and gums through brushing with a dentifrice or toothpaste, or by contacting teeth and gums by rinsing the oral cavity for about 15-90 seconds, or in the case where a lozenge, candy or chewing gum are used by sucking or chewing in the oral cavity, or in the case of a mouthspray by spraying the oral surfaces at least once weekly, or advantageously, daily.

Example 11 below shows that apigenin formulated into products for oral health, including toothpastes, gums, and lozenges (and presumably other products such as mouthwashes) can increase apigenin concentrations within saliva in an acute manner with retention seen after 1 hour and possibly much longer (as observed in the chronic user of PeroxiCare® with apigenin). Finally, this Example supports the clinical finding demonstrated in Example 10.

The anti-inflammatory, antimicrobial and anti-oxidant properties of flavonoids provide the ability to successfully treat gingivitis and periodontal disease ailments. Indeed the subject formulations enable the delivery of solubilized polyphenols including aglycone flavonoid ingredients at concentrations not achievable by currently practiced methods. Together with other desirable toothpaste formulation ingredients, polyphenols including apigenin, and optionally therapeutic fluorides, protect against plaque, gingivitis, cavities and tooth sensitivity. Together they deliver a unique, comprehensive protection to teeth.

The following Examples are illustrative, but not limiting of the compositions and methods of the invention. Other suitable modifications and adaptations of a variety of conditions and parameters normally encountered which are obvious to those skilled in the art are within the spirit and scope of the invention.

### EXAMPLES

### Example 1

### Preparation of the Apigenin/Polysorbate 80 (PS80) Concentrate

Required Ingredients Include:
9.25 grams of a highly purified PS80
0.75 grams of Apigenin powder

Procedure:
1. Add 9.25 grams of the highly purified PS80 to a 50 cc “Pyrex” beaker.
2. Add 0.75 grams of Apigenin powder to the PS80.
3. Heat the PS80/Apigenin mixture to a temperature slightly in excess of ˜275° C. At about 200° C., it will be observed that the mixture will take on a light brown/reddish color which will darken when the Apigenin is completely solubilized at ˜275° C.
4. The Apigenin/PS80 solution is set aside and allowed to cool to <100° C.

This method creates an apigenin/PS80 concentrate of approximately 5% concentration. The concentrations may be varied based upon the amount of apigenin added. One skilled in the art would understand how to, and be able to, prepare concentrates containing various amounts of active, such as 1%-5% concentrations, without undue experimentation.

### Example 2

### Apigenin Containing Toothpaste Formulation

Arm & Hammer Dental Care Advance White Breath Freshening Baking Soda Toothpaste, Frosted Mint

Active Ingredients:

Sodium Fluoride (0.24%) (Anticavity Toothpaste)

Inactive Ingredients:

Water, Baking Soda (Sodium Bicarbonate), Sorbitol, Hydrated Silica, Glycerin, Tetrasodium Pyrophosphate, Flavor, Sodium Saccharin, Cellulose Gum, Sodium Lauroyl Sarcosinate, Sodium Lauryl Sulfate, Titanium Dioxide.

TABLE II
The Composition of an Apigenin Containing
Arm & Hammer Advance White Toothpaste

| | QUANTITY | COMPOSITION |
|---|---|---|
| INGREDIENTS | (grams) | (wt. %) |
| Arm & Hammer Baking Soda | 92.0 | 92.0 |
| Advance White Toothpaste | | |
| H₂O Added | 3.4 | 3.4 |
| Polysorbate 80⁽²⁾ | 3.2 | 3.2 |
| Solubilized Apigenin ⁽¹⁾⁽⁴⁾ | 0.2 | 0.2 |
| Apigenin Powder ⁽³⁾ | 1.2 | 1.2 |
| TOTAL | 100 | 100 |

Note:
⁽¹⁾ The “Apigenin Solubilized ingredient refers to the methodology for preparing Apigenin/PS80 concentrates.
⁽²⁾Super Refined PS80 obtained from Croda Inc.
⁽³⁾ The “Apigenin Powder”, 98⁺% apigenin, Skyherb Technologies LTD, is partially solubilized in the alkaline toothpaste formulation.
⁽⁴⁾The apigenin content per gram of toothpaste is >25 times that of the Koo formulations required to inhibit the glucosyltransferase enzyme.

100 Grams of the Arm & Hammer Apigenin Containing Formulation was Prepared as Follows:
1. 92.0 grams of the Arm & Hammer white colored toothpaste was added to a 300 ml Pyrex glass beaker. The pH of the toothpaste was initially determined via pH indicator strips to be decidedly alkaline at approximately >9.5 but slightly <10.
2. 3.4 grams of a previously prepared Polysorbate/Apigenin concentrate containing 0.2 grams of Apigenin dissolved in 3.2 grams of PS80 was added to Step 1. The combined mixture was thoroughly stirred until a uniform light yellow blend was observed.
3. 3.4 gram of distilled water was added to the mixture from Step 2 and the resulting mixture thoroughly stirred to obtain a uniform blend
4. 1.2 grams of apigenin powder was added to the mixture from Step 3 and stirred until a uniform bled was obtained. The resulting mixture was a distinct yellow color—indicative of the solubilizing of apigenin as a sodium salt.

One skilled in the art would understand how to, and be able to, prepare toothpastes containing various amounts of active, such as 1%-20% concentrations, without undue experimentation.

### Example 3

### Luteolin Containing Toothpaste Formulation

Colgate Whitening Tartar Control Plus a Whitening Fluoride Toothpaste, Crisp Mint

Active Ingredients:

Sodium Fluoride (0.24%) (0.15% w/v Fluoride Ion)

Inactive Ingredients:

Sorbitol, Water, Hydrated Silica, Glycerin, PEG 12, Pentasodium Triphosphate, Tetrasodium Pyrophosphate, Sodium Lauryl Sulfate, Flavor, Sodium Hydroxide, Sodium Saccharin, Cellulose Gum, Carrageenan (Red Seaweed), Titanium Dioxide.

TABLE III
The Composition of a Luteolin Containing “Colgate
Tartar Protection” Toothpaste

| | QUANTITY | COMPOSITION |
|---|---|---|
| INGREDIENTS | (grams) | (wt. %) |
| Colgate Tarter Protection | 92.0 | 92.0 |
| Whitening Toothpaste | | |
| H₂O Added | 3.4 | 3.4 |
| Polysorbate 80⁽²⁾ | 3.2 | 3.2 |
| Solubilized Luteolin ⁽¹⁾ | 0.2 | 0.2 |
| Luteolin Powder ⁽³⁾ | 1.2 | 1.2 |
| TOTAL | 100 | 100 |

Note:
⁽¹⁾ The “Solubilized Luteolin” ingredient refers to the methodology for preparing Luteolin/PS80 concentrates.
⁽²⁾Super Refined PS80 obtained from Croda Inc.
⁽³⁾ The “Luteolin Powder”, 98⁺% luteolin, Skyherb Technologies LTD, is partially solubilized in the alkaline toothpaste formulation.

100 Grams of the Whitening Luteolin Containing Formulation was Prepared as Follows:
1. 92.0 grams of the Colgate whitening toothpaste was added to a 300 ml Pyrex glass beaker. The pH of the toothpaste was initially determined via pH indicator strips to be decidedly alkaline at approximately >9.5 but slightly <10.
2. 3.4 grams of a previously prepared Polysorbate 80/Luteolin concentrate containing 0.2 grams of Luteolin dissolved in 3.2 grams of PS80 was added to Step 1. The combined mixture was thoroughly stirred until a uniform light yellow blend was observed.
3. 3.4 gram of distilled was added to the mixture from Step 2 and the resulting mixture thoroughly stirred to obtain a uniform blend
4. 1.2 grams of luteolin powder was added to the mixture from Step 3 and stirred until a uniform bled was obtained. The resulting mixture was a distinct yellow color—indicative of the solubilizing of luteolin as a sodium salt.

One skilled in the art would understand how to, and be able to, prepare toothpastes containing various amounts of active, such as 1%-20% concentrations, without undue experimentation.

### Example 4

### Resveratrol Containing Whitening Toothpaste Formulation

Crest Complete Multi-Benefit Toothpaste, Whitening Plus Scope, Minty Fresh Stripe Toothpaste Formulation

Active Ingredients:

Sodium Fluoride (0.243%) (0.15% w/v Fluoride Ion) (Anticavity Toothpaste)

Inactive Ingredients:

Sorbitol, Water, Hydrated Silica, Disodium Pyrophosphate, Sodium Lauryl Sulfate, Flavor, Sodium Hydroxide, Alcohol (0.7%), Xanthan Gum, Sodium Saccharin, Glycerin, Carbomer 956, Cellulose Gum, Polysorbate 80, Sodium Benzoate. Cetyl Pyridinium Chloride, Benzoic Acid, Titanium Dioxide, Blue 1 Lake (CI 42090), Yellow 5 Lake.

TABLE IV
The Composition of a Resveratrol Containing Crest Complete
Multi-Benefit, Whitening Plus Scope Toothpaste

| | QUANTITY | COMPOSITION |
|---|---|---|
| INGREDIENTS | (grams) | (wt. %) |
| Crest Complete Multi-Benefit, | 98.6 | 98.6 |
| Whitening Plus Scope Toothpaste | | |
| Formulation | | |
| NaOH Crystals | 0.1 | 0.1 |
| Resveratrol Powder ⁽¹⁾⁽²⁾ | 1.2 | 1.2 |
| Citric Acid Crystals | 0.1 | 0.1 |
| TOTAL | 100 | 100 |

Note:
⁽¹⁾ The “Resveratrol Powder”, 98⁺% resveratrol, Pure Bulk Inc., is completely solubilized in the alkaline toothpaste formulation.
⁽²⁾Resveratrol was solubilized as a sodium salt and/or nano-emulsion within the toothpaste formulation.

100 Grams of the Formulation was Prepared as Follows:
1. 98.6 grams of the toothpaste was added to a 300 ml Pyrex glass beaker. The pH of the Crest toothpaste was initially determined via pH indicator strips to be decidedly alkaline at approximately 8.5.
2. About 0.1 grams of fine NaOH crystals and 1.2 grams of resveratrol powder were added to the toothpaste and thoroughly mixed into the toothpaste. A uniformly light green blend resulted with a measured pH of ˜10. The elevated alkalinity of the blended mixture resulted in the solubilizing of resveratrol as its sodium salt.
3. The pH of the mixture from Step 2 was adjusted to a pH 8.5 by the addition of about 0.1 grams of citric acid crystals.

One skilled in the art would understand how to, and be able to, prepare toothpastes containing various amounts of active without undue experimentation.

### Example 5

### Resveratrol Containing Mouthwash Formulation

Active Ingredients

### Thymol (0.064%), Eucalyptol (0.092%), Methyl Salicylate (0.060%), Menthol (0.042%)

Inactive Ingredients

### Water, Alcohol (26.9%). Benzoic Acid, Poloxamer 407, Sodium Benzoate, Caramel

The antiseptic mouthwash formulation LISTERINE® rapidly penetrates the biofilm to kill plaque and gingivitis germs.

TABLE V
The Composition of a Resveratrol Containing
LISTERINE ORIGINAL MOUTHRINSE

| | QUANTITY | COMPOSITION |
|---|---|---|
| INGREDIENTS | (grams) | (wt. %) |
| Listerine ® Original A | 98.4 | 98.4 |
| Mouthrinse Formulation | | |
| NaOH Crystals | 0.2 | 0.2 |
| Resveratrol Powder ⁽¹⁾⁽²⁾ | 1.2 | 1.2 |
| Citric Acid Crystals | 0.2 | 0.2 |
| TOTAL | 100 | 100 |

Note:
⁽¹⁾ The “Resveratrol Powder”, 98⁺% resveratrol, Pure Bulk Inc., is completely solubilized in an alkaline mouthrinse formulation.
⁽²⁾Resveratrol was solubilized as a sodium salt and/or nano-emulsion within the mouthrinse formulation.

100 Grams of the Listerine® Resveratrol Containing Mouth Rinse Formulation was Prepared as Follows:
1. 98.4 grams of the Listerine® caramel colored mouth rinse was added to a 300 ml Pyrex glass beaker. The pH of the Listerine® caramel colored mouth rinse was initially determined via pH indicator strips to be decidedly acidic at approximately 4.5.
2. 1.2 grams of Resveratrol powder was added to the solution of Step 1.
3. About 0.2 grams of fine NaOH crystals were added to the mixture from Step 2 and thoroughly mixed. A uniformly caramel colored blend resulted with a measured pH of ˜10. The elevated alkalinity of the blended mixture resulted in the solubilizing of resveratrol as its sodium salt.
4. The pH of the mixture from Step 3 was adjusted to a pH 6.5 by the addition of about 0.2 grams of citric acid crystals. A transparent solution resulted with a resveratrol concentration of 1.2 mg/ml.

### Example 6

### Luteolin Containing Mouthwash Formulation

Active Ingredients

### Thymol 0.064%, Eucalyptol 0.092%, Methyl Salicylate 0.060%, Menthol 0.042%

Inactive Ingredients

### Water, Alcohol (26.9%), Benzoic Acid, Poloxamer 407, Sodium Benzoate, Caramel

LISTERINE® Antiseptic mouth rinse rapidly penetrates the biofilm to kill plaque and gingivitis germs.

TABLE VI
The Composition of a Luteolin Containing
“LISTERINE ® ORIGINAL”
MOUTH RINSE

| | QUANTITY | COMPOSITION |
|---|---|---|
| INGREDIENTS | (grams) | (wt. %) |
| Listerine ® Original Mouthrinse | 98.3 | 98.3 |
| NaOH Crystals | 0.1 | 0.1 |
| Luteolin Powder ⁽¹⁾⁽²⁾ | 1.5 | 1.5 |
| Citric Acid Crystals | 0.1 | 0.1 |
| TOTAL | 100 | 100 |

Note:
⁽¹⁾ The “Luteolin Powder”, 98⁺% luteolin, Skyherb Technologies LTD, is solubilized in the alkaline mouthrinse formulation
⁽²⁾Luteolin was solubilized as a sodium salt

1. 98.6 grams of the Listerine® caramel colored mouthrinse was added to a 300 ml Pyrex glass beaker. The pH of the Listerine® caramel colored mouthrinse was initially determined via pH indicator strips to be decidedly acidic at approximately 4.5.
2. 1.2 grams of Luteolin powder was added to the solution of Step 1.
3. About 0.2 grams of fine NaOH crystals were added to the mixture from Step 2 and thoroughly mixed. A uniformly caramel colored blend resulted with a measured pH of ˜10. The elevated alkalinity of the blended mixture resulted in the solubilizing of luteolin as its sodium salt.
4. The pH of the mixture from Step 2 was adjusted to a pH 8 by the addition of about 0.2 grams of citric acid crystals. A transparent solution resulted with a luteolin concentration of 1.5 mg/ml.

### Example 7

### An Apigenin Containing Chewing Gum Formulation

Ingredients:

Sorbitol, Gum Base, Glycerol, Natural and Artificial flavors; Less than 2% of: Hydrogenated Starch Hydrolysate, S, Lecithin, Aspartame-Acesulfame, Mannitol, Citric Acid, Aspartame, Malic Acid, Sucralose, Acesulfame K, Colors (Yellow 5 Lake, Blue 1 Lake), BHT (To maintain freshness).

TABLE VII
The Composition of an Apigenin Containing
Extra Spearmint Chewing Gum

| | QUANTITY | COMPOSITION |
|---|---|---|
| INGREDIENTS | (grams) | (wt. %) |
| 2 Sticks of Extra Spearmint Gum | 5.93 | 95.95 |
| Polysorbate 80⁽²⁾ | 0.2375 | 3.84 |
| Solubilized Apigenin ⁽¹⁾ | 0.0125 | 0.21 |
| TOTAL | 6.18 | 100.00 |

Note:
⁽¹⁾ The “Apigenin Solubilized” ingredient refers to the methodology for preparing Apigenin/PS80 concentrates; (See Example 1).
⁽²⁾Super Refined PS80 obtained from Croda Inc.

6.18 Grams of the Extra Spearmint Apigenin Containing Formulation was Prepared as Follows:
1. 0.25 grams of a previously prepared Polysorbate 80/Apigenin liquid concentrate (See Example 1) containing 0.0125 grams of apigenin dissolved in 0.2375 grams of PS80 was added to 2 sticks of the Extra Spearmint Chewing Gum weighing 5.93 grams.
2. The Polysorbate 80/Apigenin liquid concentrate was readily and easily kneaded into the gum. The gum's physical structure and chewing quality was not noticeably altered. Significantly, the original light green/yellow color of the formulation was not visibly altered by the addition of the PS80/Apigenin concentrate.

The total quantity of dissolved apigenin computes to ˜12 mg for two sticks of gum which far exceeded the literature quantity of apigenin required to inhibit the activity of surface-bound glucosyltransferase to hinder microbial glucan-forming activity. In addition, the solubilized apigenin contributes to dental health via its anti-inflammatory and anti-oxidant properties. Apigenin's role inhibiting and/or stopping the accumulation of microorganisms contributes to the prevention of dental plaque, dental caries, gingivitis and other oral problems. Further the nonionic PS80 surfactant can further serve to aid in apigenin contact with the surface structure of the plaque.

The gum's composition of the present invention can be combined with effective amounts of other components, such as other aglycone flavonoids.

### Example 8

### Formation of Soluble Flavone Salt Formulations within an Alkaline Toothpaste Comprised of Sodium Bicarbonate, Peroxide and a Peroxide Stabilizer at Concentrations Up to 17.5%

Arm & Hammer PeroxiCare® Toothpaste Formulation

Active Ingredient:

Sodium Fluoride (0.24%)

Inactive Ingredients:

Sodium Bicarbonate (Baking Soda), PEG-8, PEG/PEG-116/66 Copolymer, Tetrasodium Pyrophosphate, Sodium Carbonate Peroxide, Silica, Sodium Saccharin, Flavor, Water, Sodium Lauryl Sulfate, Sodium Lauryl Sarcosinate.

TABLE VIII
The Composition of Alkaline Soluble Sodium
Apigenin Salt Containing Toothpaste

| | QUANTITY | COMPOSITION |
|---|---|---|
| INGREDIENTS | (grams) | (wt. %) |
| PeroxiCare ® Toothpaste | 150 | 99.6 |
| Formulation | | |
| Solubilized Apigenin Powder ⁽¹⁾⁽²⁾ | 0.6 | 0.4 |
| TOTAL | 150.6 | 100.00 |

Note:
⁽¹⁾ The Solubilized Apigenin ingredient refers to the formation of an alkaline sodium apigenin salt formed when apigenin added to the PeroxiCare ® toothpaste formulation.
⁽²⁾The “Apigenin Powder”, 98+% apigenin, Skyherb Technologies LTD, is partially solubilized in the alkaline toothpaste formulation.

BASIS: 100 cc of PeroxiCare® Toothpaste

1. Dispense 100 cc of PeroxiCare® toothpaste into a clean 200 ml Pyrex glass beaker, which has been cleaned with an ethyl alcohol rinse.
2. Verify that the weight of the 100 cc of the PeroxiCare® toothpaste from Step 1 is 150 grams. If not, add or remove PeroxiCare® to achieve a weight of 150 grams.
3. Confirm that the pH of the PeroxiCare® from Step 2 is ≧10. The pH is determined by taking about <0.1 grams of the toothpaste formulation PeroxiCare® and thoroughly mixing it with ˜20 cc of distilled water contained within a 30 ml Pyrex glass beaker. A drop of this liquid mixture is then added to a strip of pH paper to determine the pH. Allow at least a minute of contact with the pH before comparing the color change to determine the pH. In the unlikely event that the pH is <10, the addition of sodium hydroxide crystals to the toothpaste formulation can be made to achieve a pH level˜10.
4. Add 600 mg of apigenin powder to the mixture from Step 2 and thoroughly mix with a spatula for at 5 minutes to obtain a uniform yellow mixture.
5. The mixture from Step 4 containing 6 mg/ml of a sodium apigenin salt within a PeroxiCare®mixture is then poured into suitable airless dispensing tubes.

Additional PeroxiCare® toothpaste samples at various apigenin concentrations were prepared as noted in Table IX. The HPLC results shown in Table IX verify that increasing the apigenin content up to 17.5% does result in significantly increased soluble apigenin concentrations.

One skilled in the art would understand how to, and be able to, prepare toothpastes containing various amounts of active, such as 0.1%-17.5% concentrations, without undue experimentation.

TABLE IX
Apigenin content of PeroxiCare ® formulated toothpastes

| Products (%) | Apigenin (μg/g) |
|---|---|
| 0.2 | 1,543.0 |
| 0.5 | 1,868.1 |
| 1.0 | 4,101.4 |
| 5.0 | 15,014.0 |
| 17.5 | 66,287.0 |

### Example 9

### The Treatment of Oral Inflammatory and Microbacterial Disorders by the Application of Soluble Sodium Apigenin Salt Formulations within an Alkaline Toothpaste Comprised of Sodium Bicarbonate, Sodium Carbonate Peroxide and a Peroxide Stabilizer

Ten patients had gingivitis diagnosed by bleeding on probing and gingival index. These patients used the toothpaste of Example 8 without flossing, rinse or professional care for two weeks. Each patient demonstrated significant improvement with little or no bleeding on probing and a healthier gingival index. The product successfully treated gingivitis for this patient population.

### Example 10

### A Double Blind, Randomized Trial Comparing Efficacy of an Alkaline Apigenin Dentifrice for the Treatment of Gingivitis

Primary Objective:

The primary objective of this study was to evaluate efficacy of alkaline apigenin toothpaste similar to that described in Example 8 for topical treatment of gingivitis.

Overall Study Design:

This study was a randomized, double blind trial clinical comparing alkaline toothpaste with apigenin for the treatment of gingivitis. Twenty subjects were enrolled with varying severity of gingivitis, if not early periodontitis. Each subject had gingiva around at least 4 teeth with an index of 2, moderate inflammation, redness, edema and bleeding as described by Loe and Silness. Recorded clinical parameters included the following: (1) Gingival Index; (2) Bleeding Index (number of surfaces bleeding and severity by number of teeth present); (3) Periodontal Pocket Depths, and (4) Plaque Index.

A screening and examination visit took place at the study center where intraoral photographs of the selected subjects were taken. Ten subjects used the alkaline PeroxiCare® toothpaste, and 10 subjects used the alkaline toothpaste with apigenin exclusively for 2 weeks with no rinsing or flossing. Selected subjects were instructed to use only the test material provided for 2 minutes twice a day (morning and before bed) and given instruction in sulcular brushing technique and told to use a two-minute timer. Each subject was also shown the appropriate amount of test material to be put on the new brush that they were given. Each subject was instructed to brush each quadrant of the mouth for 30 seconds making a total brushing cycle of 2 minutes. Subjects were also instructed to keep a daily diary to further document their experience with the test material

The clinical parameters of Gingival Index, Bleeding Index, Periodontal Pocket Depth and Plaque Index were recorded at day 1 and day 15. Intraoral photographs of teeth involved were taken on both days and the diaries were reviewed in order to collect all necessary information.

Toothpaste Preparation:

Toothpastes used in this study were prepared by the procedures described in Example 8. Food grade dyes were utilized so that the 2 formulations had the same color so neither the investigator nor patient knew which formulation they received. The 2 formulations were labeled B and C. The formulator who prepared the B and C products was the only person who knew the compositions of the formulations as noted in the Table X.

TABLE X
Toothpaste Sample Compositions

| | ⁽¹⁾Alkaline | | | |
|---|---|---|---|---|
| Sample | Toothpaste | Apigenin | TOTAL | |
| Identification | (Wt. %) | (Wt. %) | (Wt. %) | pH |
| B | 100 | 0 | 100 | ~10.2 |
| Alkaline | | | | |
| Toothpaste | | | | |
| C | 99.3 | 0.7 (10 mg/ml) | 100 | ~10.2 |
| Alkaline | | | | |
| Toothpaste | | | | |
| with Apigenin | | | | |

Note:
⁽¹⁾The Alkaline Toothpaste ingredients are listed in Example 8

Study Evaluations

Gingival Index (0-3 Point Scale):

As described by Loe and Silness:

Score 0—Normal;

Score 1—Mild inflammation, slight color change, no bleeding;

Score 2—Moderate inflammation, edema redness bleeding on probing;

Score 3—Severe inflammation redness, edema, easy bleeding.

The final gingival index score equals summation of the scores per surface/total number of surfaces (4 per tooth). These scores were then added for the four teeth involved in the study.

Bleeding Index: (0-5) Point Scale:

An early sign of gingivitis is bleeding on probing and, in 1971; Muhlemann and Son described the Sulcus Bleeding Index (SBI). The criteria for scoring are as follows:

Score 0—healthy looking papillary and marginal gingiva no bleeding on probing;

Score 1—healthy looking gingiva, bleeding on probing;

Score 2—bleeding on probing, change in color, no edema;

Score 3—bleeding on probing, change in color, slight edema;

Score 4—bleeding on probing, change in color, obvious edema;

Score 5—spontaneous bleeding, change in color, marked edema

Periodontal Pocket Depth:

Depth measurements in mm were taken with a periodontal probe at 6 points around each tooth. These scores were added for the four teeth chosen in the study.

Results

Both the alkaline toothpaste and alkaline toothpaste with apigenin formulations resulted in substantial improvements for the 2 week period study duration for all measured scores as noted in Table XI. Significantly, the alkaline toothpaste with apigenin resulted in improvements of 3.1%, 34.2% and 30.6% of the Pocket Depth, Bleeding and Gingival Indexes, respectively when compared to the alkaline toothpaste formulation without apigenin.

The anti-inflammatory and antimicrobial properties of apigenin contributed to the improvements in the periodontal and gingival health noted in this study.

Some subjects noted that the addition of apigenin to the alkaline toothpaste improved the taste and flavor of the formulation.

TABLE XI
A Comparison of the ⁽¹⁾Alkaline Toothpaste and
Alkaline Toothpaste with Apigenin Improvements

| | POCKET | BLEEDING | GINGIVAL |
|---|---|---|---|
| | DEPTH | INDEX | INDEX |
| SAMPLE | (% | (% | (% |
| IDENTIFICATION | Improvement) | Improvement) | Improvement) |
| Alkaline Toothpaste | 10.31 | 21.82 | 10.76 |
| Alkaline Toothpaste | 10.63 | 29.82 | 14.05 |
| with Apigenin | | | |

(% Improvements after Morning & Evening Brushing Teeth for 2 Weeks)
Note:
⁽¹⁾The Alkaline Toothpaste ingredients are listed in Example 8

### Example 11

### Apigenin Content of Formulated Products and of Apigenin in Saliva and Sulcus Following Product Use

Primary Objectives:

The primary objective of this study was to evaluate the retention of apigenin in the saliva and sulcus fluid when using toothpastes of the invention.

Overall Study Design:

Six subjects provided saliva samples with 5 different apigenin containing toothpastes: (1) before brushing, (2) after brushing without rinsing, (3) immediately after rinsing, and (4) one hour after rinsing. Each subject brushed their teeth for 2 minutes using the sulcular brushing technique with ˜1.8 g toothpaste on a new toothbrush. One subject (chronic user) followed the brushing routine for two minutes, twice daily, morning and night, for more than sixty days prior to the start of the study.

Four (4) different sulcus locations were measured using absorbent paper points. The infiltrated paper points were collected after brushing and 1 hour post rinsing. All determinations of apigenin in saliva and sulcus fluid were conducted in a manner blind to the treatment assignments.

Sample Preparations:

Toothpaste samples were prepared by the procedures described in the “Methods of Preparing Formulations of the Invention Section” and Examples 1, 3&8.

Details of the 5 toothpaste samples utilized in this study are provided in Table XII.

TABLE XII
Toothpaste Sample Characteristics

| | Apigenin | | | |
|---|---|---|---|---|
| TOOTH- | Content | Apigenin | | Number of |
| PASTE | (mg/ml) | Form | pH | Subjects |
| PeroxiCare ® | 8 | Sodium | ~10.5 | 1 acute use |
| | | Salt | | 1 chronic use |
| ⁽²⁾Crest | 10 | Sodium | ~10.5 | 1 |
| Pro-Health | | Salt | | |
| Crest | 2 | PS80 | ~6.0 | 1 |
| Pro-Health | | Concentrate | | |
| Crest | 10 | Nano | ~6.0 | 1 |
| Pro-Health | | particulates | | |
| Tom's of | ⁽¹⁾ N/A | — | ~8.0 | 1 |
| Maine | | | | |
| Propolis & | | | | |
| Myrrh | | | | |

Note:
⁽¹⁾ Propolis presumably contains a low apigenin concentration
⁽²⁾Modified by the addition of NaOH

The ingredients contained within each of the toothpaste formulations are listed in Table XIII.

TABLE XIII
Toothpaste Sample ingredients

| Toothpaste | Active | |
|---|---|---|
| Type | Ingredients | Inactive Ingredients |
| PeroxiCare ® | Sodium | Sodium Bicarbonate (Baking Soda), |
| | Fluoride | PEG-8, PEG/PEG-116/66 Copolymer, |
| | (0.24%) | Tetrasodium Pyrophosphate, Sodium |
| | | Carbonate Peroxide, Silica, Sodium |
| | | Saccharin, Flavor, Water, Sodium |
| | | Lauryl Sulfate, Sodium Lauryl |
| | | Sarcosinate. |
| Crest | Stannous | Glycerin, Hydrated Silica, Sodium |
| Pro-Health | Fluoride | Hexametaphosphate, Propylene Glycol, |
| | (0.454%) | PEG 6, Water, Zinc Lactate, Flavor, |
| | | Trisodium Phosphate, Sodium Gluconate, |
| | | Sodium Lauryl Sulfate, Sodium |
| | | Saccharin, Carrageenan, Stannous |
| | | Chloride, Xanthan Gum, Polyethylene, |
| | | Titanium Dioxide, Blue 1 Lake, Blue 1 |
| Tom's of | — | Calcium Carbonate, Water, Glycerin, |
| Maine | | Sodium Bicarbonate, Xylitol, |
| Propolis & | | Carrageenan, Fennel Oil, Sodium Lauryl |
| Myrrh | | Sulfate, Myrrh Resin Extract, Propolis |
| | | Extract |

Experimental Methods:

Apigenin in the toothpaste products was extracted using a liquid-liquid protocol. Briefly, 0.5 gm of toothpaste was mixed with 5 ml H₂O. After 2 minutes of rigorous vortexing, apigenin in the resulting mixture was extracted with 15 ml ethyl acetate. After 2 minutes of rigorous vortexing and 15 minutes of centrifugation, aliquots of the ethyl acetate and water fractions were dried under N₂ gas and reconstituted for apigenin analysis using HPLC-ECD. The concentration of apigenin in toothpaste products is the sum of the apigenin content in both water and ethyl acetate fractions.

Saliva was collected at each time point in 50 ml Falcon tubes and immediately transferred to a freezer. After collection, paper points infiltrated with sulcus fluid were also placed in 50 ml and stored in a freezer. All samples were transported to Tufts University for analysis within 8 hours of collection.

Apigenin in saliva and sulcus was quantified using a high performance liquid chromatograph (HPLC) with electrochemical detection (ECD). Apigenin in 1 ml saliva or 4 paper points containing sulcus fluid was extracted using 3 ml acetonitrile. After 2 minutes of rigorous vortexing, the mixture was spun at 2,000 rpm for 15 minutes at 4° C. The supernatant was dried under N₂ gas, reconstituted with mobile phase A, and analyzed for apigenin with HPLC-ECD. The limit of quantification (LOQ) for this assay is 15 ng/ml saliva.

The concentration of apigenin in saliva, paper points, and the toothpaste products was calculated using a standard curve constructed with authenticated apigenin. The standard curve was linear with R² value of 0.9973.

Results

The analytical apigenin concentrations in the toothpaste test samples are itemized in the Table XIV.

TABLE XIV
The Analytical Determined Apigenin Content of the Toothpastes

| | | | | | Normalized |
|---|---|---|---|---|---|
| | Apigenin | | | Apigenin | Apigenin |
| TOOTH- | Content | Apigenin | | Content | Content to |
| PASTE | (mg/ml) | Form | pH | (μg/g) | 1 mg/ml |
| PeroxiCare ® | 8 | Sodium | ~10.5 | 5603 | 700.4 |
| | | Salt | | | |
| ⁽²⁾Crest | 10 | Sodium | ~10.5 | 7964 | 797.4 |
| Pro-Health | | Salt | | | |
| Crest | 2 | PS80 | ~6.0 | 1799 | 899.5 |
| Pro-Health | | Concen- | | | |
| | | trate | | | |
| Crest | 10 | Nano | ~6.0 | 7609 | 760.9 |
| Pro-Health | | Particu- | | | |
| | | lates | | | |
| Tom's of | ⁽¹⁾ N/A | — | ~8.0 | 0.23 | 0.04 |
| Maine | | | | | |
| Propolis & | | | | | |
| Myrrh | | | | | |

Note:
⁽¹⁾ Propolis presumably contains a low apigenin concentration
⁽²⁾Modified by the addition of NaOH

The analytical measured apigenin content in saliva and sulcus fluid of toothpaste subjects as a function of time are listed in Table XV.

TABLE XV
The Analytical Determined Apigenin Content in the
Saliva and Sulcus Fluids of Toothpaste Subjects

| | | SALIVA⁽⁴⁾ | | | | SULCUS⁽⁵⁾ | |
|---|---|---|---|---|---|---|---|
| | | TIME POINTS | | | | | |
| | | Apigenin Content ⁽¹⁾ | | | | | |
| | Apigenin | (μg/ml) | | | | | |
| | Content | ⁽¹⁾ (μg) | | | | | |
| TREATMENT | (mg/ml) | 1 | 2 | 3 | 4 | 1 | 2 |
| Crest | 10 | ND⁽³⁾ | 679.76 | 8.1 | 0.09 | 0.41 | 0.17 |
| Pro-Health | | | (67.98) | (0.81) | (0.01) | (0.04) | (0.02) |
| Nano- | | | | | | | |
| Particulates | | | | | | | |
| PeroxiCare ® | 8 | 0.72 | 235.00 | 26.00 | 3.29 | 0.28 | 0.04 |
| Sodium | | (0.09) | (29.38) | (3.25) | (0.41) | (0.04) | (0.01) |
| Apigenin | | | | | | | |
| Salt | | | | | | | |
| (Chronic Use) | | | | | | | |
| ⁽⁶⁾Crest | 10 | ND⁽³⁾ | 459.00 | 8.72 | 3.97 | 0.80 | 0.19 |
| Pro-Health | | | (45.90) | (0.87) | (0.40) | (0.08) | (0.02) |
| Sodium | | | | | | | |
| Apigenin | | | | | | | |
| Salt | | | | | | | |
| PeroxiCare ® | 8 | ND⁽³⁾ | 119.00 | 37.40 | 0.96 | 0.21 | 0.07 |
| Sodium | | | (14.88) | (4.66) | (0.12) | (0.03) | (0.01) |
| Apigenin | | | | | | | |
| Salt | | | | | | | |
| (Acute Use) | | | | | | | |
| Crest | 2 | ND⁽³⁾ | 29.10 | 8.08 | 0.14 | 0.15 | 0.02 |
| Pro-Health | | | (14.05) | (4.04) | (0.07) | (0.08) | (0.01) |
| PS80/ | | | | | | | |
| Apigenin | | | | | | | |
| Concentrate | | | | | | | |
| Tom's of | N/A | ND⁽³⁾ | ND⁽³⁾ | ND⁽³⁾⁾ | ND⁽³⁾ | ND⁽²⁾ | ND⁽²⁾ |
| Maine | | | | | | | |
| Propolis & | | | | | | | |
| Myrrh | | | | | | | |

Note:
⁽¹⁾ Values in Parenthesis normalized to 1 mg/ml apigenin in the toothpaste formulations.
⁽²⁾Apigenin in the sulcus fluid cannot be expressed as μg/ml because the fluid volume in the paper points is not known.
⁽³⁾ND, Not Detected.
⁽⁴⁾Saliva Time Points: (1) before brushing, (2) after brushing without rinsing, (3) immediately after rinsing, and (4) one hour after rinsing.
⁽⁵⁾Sulcus Time Points: (1) after brushing, (2) 1 hour post rinsing.
⁽⁶⁾Modified by the addition of NaOH.

Apigenin was determined in each toothpaste product and ranged from 0.23 μg/g (Propolis containing toothpaste) to 7964 μg/g (A Modified Acidic Formulation with apigenin salt). In each toothpaste test, the applied amount was ˜1.8 g. With the exception of the chronic alkaline formulation user, no toothpaste subject had a detectable saliva concentration of apigenin at baseline. The sulcus fluid collected immediately after brushing was consistently higher in apigenin than 1 hour later though, importantly, it was still present at this second time point. The apigenin increased markedly immediately after brushing and without rinsing with concentrations ranging from 29.10 to 679.76 μg/ml.

The subject using alkaline formulation with apigenin on a chronic basis had a detectable amount of apigenin (0.72 μg/ml) 12 hours after the last use of the product (i.e., at baseline).

The acidic formulation has a pH 6 and alkaline formulation a pH 10. Salivary apigenin was highest after brushing and 1 hour after rinsing with the modified alkaline apigenin salt but was higher immediately after rinsing with alkaline toothpaste formulation. The various formulations tested demonstrate that different solubilizing technologies can result in therapeutically effective concentrations of bioavailable aglycone flavonoids in the oral cavity both at near, medium and long term time durations. Differing formulations and concentrations can be used to achieve the desired concentrations levels.

### Example 12

### Saliva Concentrations of Chronic 1% Apigenin Toothpaste Users

Study Design

Two subjects who have used PeroxiCare® with apigenin at a 1% concentration on a chronic basis provided saliva samples at:

(i) pre-brush,

(ii) 1-hour after brushing, and

(iii) 8-hours after brushing.

All determinations of apigenin in saliva were conducted in a manner blind to the treatment assignments.

Methods

Saliva was collected at each time point in 50 ml Falcon tubes and immediately transferred to a freezer. All samples were transported to Tufts University within 24 hours of collection. Apigenin in saliva was quantified using high performance liquid chromatograph (HPLC) with electrochemical detection (ECD). Apigenin in 1 ml saliva was extracted using 3 ml acetonitrile. After 2 min of rigorous vortexing, the mixture was spun at 2,000 rpm for 15 min at 4° C. Supernatant was dried under N₂ gas, reconstituted with mobile phase A, and analyzed for apigenin with HPLC-ECD. The limit of quantification (LOQ) for apigenin and EGCG in this assay is 15 and 25 ng/ml saliva, respectively. Table XVI shows analytical analysis of the apigenin saliva levels in chronic users.

TABLE XVI
SALIVA LEVELS OF APIGENIN IN CHRONIC USERS

| Chronic Use | Pre-brush | 1-h Post-brush | 8-h Post-brush |
|---|---|---|---|
| PeroxiCare ® - | 0.29 | 26.2 | 3.9 |
| 1% Apigenin, - #1 | | | |
| PeroxiCare ® - | 0.12 | 1.95 | 0.0 |
| 1% Apigenin, - #2 | | | |

Conclusion

The results from this clinical study show that in chronic users, apigenin levels can persist in the saliva for at least 8 hours post-brushing with the potential for longer term benefits being seen as concentrations and frequencies are increased.

### Example 13

### Apigenin Content in Saliva Following the Use of Gum or Lozenge Containing Apigenin Salts

Primary Objectives

The primary objective of this study was to evaluate the retention of apigenin in the saliva when using a gum or lozenge of the invention.

Overall Study Design

The subjects testing the gum (n=1) and lozenge (n=1) provided saliva samples before and 5 minutes after use of the products but no sulcus fluid was collected. All determinations of apigenin in saliva were conducted in a manner blind to the treatment assignments.

Sample Preparations

Gum samples were prepared by infusing a Spearmint Sugarfree Gum by forming a molten mixture of the gum to about 60° C. and adding a few crystals NaOH crystals to achieve a pH of ˜10.0. Apigenin powder was then added to form the soluble sodium salt of Apigenin (˜0.5 wt. %). Additionally, Xylitol ((˜0.5 wt. %) was added to mixture. The molten mixture was then poured into spherical shapes. When cooled, the modified gum was coated with a thin film of Xylitol crystals.

The lozenges were prepared for gummy bears infused with the sodium salt of Apigenin and coated with xylitol crystals. The gummy bears were heated within a microwave oven such that a molten mixture was formed ˜65° C. A concentrated NaOH aqueous concentrate was added to the molten gummy bears to achieve an alkaline pH˜10.0. Apigenin powder was then added to the mixture to form a 1 wt. % of the soluble sodium Apigenin salt. The molten mixture was the poured into ˜½″ diameter ball shapes. When cooled, the resulting modified gummy bear shapes were coated with a thin layer of xylitol.

Experimental Methods

The gum and lozenge samples were pulverized in liquid nitrogen. Apigenin in 0.5 gm of the resulting powder was determined by the same protocol described in Example 11.

Results

The analytical apigenin concentrations in the gum and lozenge test samples are itemized in Table XVII.

TABLE XVII
The Analytical Determined Apigenin Content of the Test Samples

| | | | | | Normalized |
|---|---|---|---|---|---|
| | Apigenin | | | Apigenin | Apigenin |
| | Content | Apigenin | | Content | Content to |
| PRODUCT | (mg/ml) | Form | pH | (μg/g) | 1 mg/ml |
| Gum | 0.5 wt. % | Sodium | ~10.0 | 4032 | 8064 |
| (Wrigley's) | | Salt | | | (1 wt. %) |
| Lozenge | 1.0 wt. % | Sodium | ~10.0 | 7963 | 7963 |
| (Gummi Bear) | | Salt | | | (1 wt. %) |

The analytical measured apigenin content in saliva of gum and lozenge as a function of time using gum and lozenge are listed in the Table XVIII.

TABLE XVIII
The Analytical Determined Apigenin Content
in the Saliva of Gum and Lozenge Subjects

Apigenin Content (μg/ml) in Saliva
Time Points ^{(1) (2)}

| TREATMENT | 1 | 2 |
|---|---|---|
| Gum (Wrigley's) | 0.18/(0.36) | 23.36/(46.72) |
| Lozenge (Gummi Bear) | 0.09/(0.09) | 75.05/(75.05) |

Note:
⁽¹⁾ Values in parenthesis normalized to 1 wt. % concentration in the gum & lozenge.
⁽²⁾ Saliva Time Points; the subjects testing the gum and lozenge provided saliva samples (1) before and (2) 5 minutes after use.

The use of the gum and lozenge dosage forms was associated with an increase in salivary apigenin after 5 minutes. Low baseline levels of salivary apigenin were noted in the subjects testing the gum and lozenge (0.09 and 0.18 μg/ml, respectively).

### Example 14

### Epigallocatechin Gallate (EGCG) in Toothpaste Formulations and Saliva Following Use

Primary Objectives:

The primary objective of this study was to evaluate the retention of Epigallocatechin Gallate (EGCG) in toothpaste formulations and saliva.

Overall Study Design:

A saliva sample with an EGCG containing alkaline toothpaste was obtained (1) before brushing and (2) 1 minute after brushing immediately after rinsing. The subject brushed for 2 minutes using the sulcular brushing technique with ˜1.8 gm toothpaste on a toothbrush.

Sample Preparations:

A toothpaste sample was prepared by a procedure described in the “Methods of Preparing Formulations of the Invention Section” and Examples 1, 3 & 8. Details of the toothpaste sample are provided in the following Table XIX.

TABLE XIX
Toothpaste Ingredients

| ⁽¹⁾ Toothpaste | Content |
|---|---|
| Ingredients | (wt. %) |
| PeroxiCare ® | 99.8 |
| Ascorbic Acid | 0.1 |
| EGCG | 0.1 |

Note:
⁽¹⁾ A minimal amount of NaOH crystals was added to adjust the formulation to a pH of ~10.5

Experimental Method:

A 5 ml sample of saliva was collected 10 minutes prior to brushing. Tooth brushing was conducted for 2 minutes with 1.8 gm of toothpaste applied to the brush. One minute after brushing, the oral cavity was rinsed with 50 ml of water. Following rinsing, about 8 ml of saliva was collected. The pH of the saliva was measured. In addition, the saliva samples were further made alkaline with the addition of NaOH crystals and the color of the resulting sample noted.

Results:

Alkalizing the neutral saliva sample (pH˜7.0) with sodium hydroxide crystals to a pH˜10.5 resulted in a red/brown color of the saliva sample indicating that EGCG was retained after brushing. There was no color change in the saliva sample before brushing.

### Example 15

### Apigenin and Epigallocatechin Gallate in Saliva Following Use of Formulated Toothpaste Products

Study Design

This is a clinical study testing the effect of using one of 3 toothpaste products formulated with apigenin and epigallocatechin gallate (EGCG) on the retention of these flavonoids in the saliva. Briefly, 3 subjects provided saliva samples:

(i) before brushing,

(ii) after brushing and rinsing,

(iii) 1-h after brushing, and

(iv) 3-h after brushing.

Each subject brushed their teeth for 2 min using the sulcular brushing technique with ˜1.8 gm toothpaste on a new toothbrush. Each subject brushed with only one of 3 products:

(a) 15 mg/ml EGCG+15 mg/ml Apigenin micro-particulates in Crest Pro-Health
(prepared by the procedures described in Examples 2 & 3)

(b) 50 mg/ml EGCG salt+50 mg/ml Apigenin salt in the alkaline PeroxiCare®
(prepared by the procedures described in Example 8)

(c) 50 mg/ml EGCG+50 mg/ml Apigenin PS80 concentrate in Crest Pro-Health
(prepared by the procedures described in Example 31)

All determinations of apigenin and EGCG in saliva were conducted in a manner blind to the treatment assignments.

Methods

Saliva was collected at each time point in 50 mL Falcon tubes and immediately transferred to a freezer. All samples were transported to Tufts University within 8 hours of collection.

Apigenin in saliva was quantified using high performance liquid chromatograph (HPLC) with electrochemical detection (ECD). Apigenin and EGCG in 1 mL saliva was extracted using 3 ml acetonitrile. After 2 min of rigorous vortexing, the mixture was spun at 2,000 rpm for 15 min at 4° C. Supernatant was dried under N₂ gas, reconstituted with mobile phase A, and analyzed for apigenin with HPLC-ECD. The limit of quantification (LOQ) for apigenin and EGCG in this assay is 15 and 25 ng/ml saliva, respectively.

Apigenin in the toothpaste products was extracted using a liquid-liquid protocol. Briefly, 0.5 gm toothpaste was mixed with 5 mL H₂O. After 2 min of rigorous vortexing, flavonoids in the resulting mixture were extracted with 15 ml ethyl acetate. After 2 min of rigorous vortexing and 15 min of centrifugation aliquots of the ethyl acetate and water fractions were dried under N₂ gas and reconstituted for analysis using HPLC-ECD. The concentration of apigenin and EGCG in toothpaste products is the sum of the apigenin content in both water and ethyl acetate fractions.

Table XX shows that both apigenin and EGCG can exist in toothpaste samples.

TABLE XX
APIGENIN AND EGCG CONTENT OF TEST PRODUCTS⁽¹⁾

| | Apigenin | | EGCG | |
|---|---|---|---|---|
| | Tooth- | Adminis- | Tooth- | Adminis- |
| | paste | tered | paste | tered |
| Products | (μg/g) | (mg) | (μg/g) | (mg) |
| 15 mg/ml EGCG + | 1424.7 | 2.56 | 1241.3 | 2.23 |
| 15 mg/ml Apigenin | | | | |
| micro in Pro-Health | | | | |
| 50 mg/ml EGCG | 1844.1 | 3.31 | 22.7 | 0.04 |
| salt + 50 mg/ml | | | | |
| Apigenin salt in | | | | |
| PeroxiCare ® | | | | |
| 50 mg/ml EGCG + | 1356.3 | 2.44 | 343.0 | 0.62 |
| 50 mg/ml Apigenin | | | | |
| PS80 concentrate | | | | |
| in Pro-Health | | | | |

⁽¹⁾Administered doses of apigenin and EGCG were obtained based on the amount of toothpaste used by each person. The amount of toothpaste was estimated at 1.8 gm.

Table XXI shows the concentrations of apigenin in saliva.

TABLE XXI
APIGENIN CONTENT IN SALIVA

| | Time Point | | | |
|---|---|---|---|---|
| | | | 1-h | 3-h |
| Toothpaste | Pre-brush | Post-brush | Post-brush | Post-brush |
| Acute Use | μg/mL | | | |
| 15 mg/ml EGCG + | 0.0 | 45.5 | 11.0 | 0.0 |
| 15 mg/ml Apigenin | | | | |
| micro in Pro-Health | | | | |
| 50 mg/ml EGCG | 0.0 | 22.6 | 12.7 | 0.0 |
| salt + 50 mg/ml | | | | |
| Apigenin salt in | | | | |
| PeroxiCare ® | | | | |
| 50 mg/ml EGCG + | 0.0 | 16.0 | 10.2 | 0.0 |
| 50 mg/ml Apigenin | | | | |
| PS80 concentrate | | | | |
| in Pro-Health | | | | |

Table XXII shows EGCG concentrations in saliva. It is apparent that apigenin concentration remains in larger concentrations than EGCG for extended time periods.

TABLE XXII
EGCG CONTENT IN SALIVA

| | Time Point | | | |
|---|---|---|---|---|
| | | | 1-h | 3-h |
| Toothpaste | Pre-brush | Post-brush | Post-brush | Post-brush |
| Acute Use | μg/mL | | | |
| 15 mg/ml EGCG + | 0.0 | 1.4 | 0.3 | 0.0 |
| 15 mg/ml Apigenin | | | | |
| micro in Pro-Health | | | | |
| 50 mg/ml EGCG | 0.0 | 0.5 | 0.2 | 0.0 |
| salt + 50 mg/ml | | | | |
| Apigenin salt in | | | | |
| PeroxiCare ® | | | | |
| 50 mg/ml EGCG + | 0.0 | 0.5 | 0.1 | 0.0 |
| 50 mg/ml Apigenin | | | | |
| PS80 concentrate | | | | |
| in Pro-Health | | | | |

Conclusion

The results from this clinical study show that both flavone apigenin and flavan-3-ol epigallocatechin gallate (EGCG) can be successfully formulated into a toothpaste product and found in saliva not only immediately after use but also for at least one hour following. Apigenin appears to persist at higher concentrations in the saliva compared to EGCG. These data are consistent with earlier studies.

### Example 16

### The Solubility of Various Aglycone Flavonoid Classes in a PS80 Surfactant Solvent

The solubility concentrations of several aglycone flavonoids within acidic formulations are severely limited. For example, all stannous fluoride formulations which reduce gingivitis, plaque, caries etc., are decidedly acidic. Consequently, the thermal processing methods described in U.S. Pat. No. 8,637,569 were used to prepare the PS80/Aglycone Flavonoid concentrates as noted in the Table XXIII. All solubility tests were conducted with 20 ml of PS80 contained within 80 ml Pyrex glass beakers. Complete solubilization of the PS80/Aglycone Flavonoid was achieved at temperature levels exceeding 125° C. The PS80/Aglycone Flavonoid concentrates representing various classes of flavonoids can be added to a variety of oral formulations from pHs preferably ranging 3 to 11 so as to enhance the aglycone flavonoid solubility.

TABLE XXIII
The Solubility of Various Flavonoids Classes
in PS80 via the Thermal Processing Method

| | | | | | PS80 |
|---|---|---|---|---|---|
| | | PURI- | ⁽¹⁾ Water | ^{(1) (2)}PS80 | Solution |
| | CLASS/ | TY | Solubility | Solubility | Temp. |
| ⁽³⁾FLAVONOID | TYPE | (%) | (mg/ml) | (mg/ml) | (° C.) |
| Epigallo- | Flavanol | 98 | ~25 | 50 | ~150 |
| catechin | | | | | |
| Gallate | | | | | |
| (EGCG) | | | | | |
| Catechin | Flavanol | 90 | ~5 | 50 | ~210 |
| Genistein | Isoflavone | 98 | 0.12 | 50 | ~220 |
| Naringenin | Flavanone | 98 | 0.04 | 50 | ~280 |
| Chrysin | Flavone | 98 | 0.08 | 50 | ~140 |
| Diosmetin | Flavone | 98 | 0.08 | 50 | ~190 |

Note:
⁽¹⁾ Solubility values at 20° C.
⁽²⁾The Flavonoids concentrations are not the maximum solubility levels in the PS80 solvent (Tween 80, High Purity from Croda Inc.)
⁽³⁾The Flavonoids were obtained from Shaanxi Huike Botanical Development Co., Ltd, Xi'an, Shaanxi China

### Example 17

### Formation of Soluble Curcumin Salt Formulations within an Alkaline Toothpaste Comprised of Sodium Bicarbonate, Peroxide and a Peroxide Stabilizer

Arm & Hammer PeroxiCare® Toothpaste Formulation

Active Ingredient:

Sodium Fluoride (0.24%)

Inactive Ingredients:

Sodium Bicarbonate (Baking Soda), PEG-8, PEG/PEG-116/66 Copolymer, Tetrasodium Pyrophosphate, Sodium Carbonate Peroxide, Silica, Sodium Saccharin, Flavor, Water, Sodium Lauryl Sulfate, Sodium Lauryl Sarcosinate.

TABLE XXIV
The Composition of Alkaline Soluble Sodium
Curcumin Salt Containing Toothpaste

| | QUANTITY | COMPOSITION |
|---|---|---|
| INGREDIENTS | (grams) | (wt. %) |
| PeroxiCare ® Toothpaste | 150 | 99.6 |
| Formulation | | |
| Solubilized curcumin (1) | 0.6 | 0.4 |
| TOTAL | 150.6 | 100.00 |

Note:
(1) The Solubilized curcumin ingredient refers to the formation of an alkaline sodium curcumin salt formed when curcumin added to the PeroxiCare ® toothpaste formula

Basis: 100 cc of PeroxiCare® Toothpaste

1. Dispense 100 cc of PeroxiCare® toothpaste into a clean 200 ml Pyrex glass beaker which has been cleaned with an ethyl alcohol rinse.
2. Verify that the weight of the 100 cc of the PeroxiCare® toothpaste from Step 1 is 150 grams. If not, add or remove PeroxiCare® to achieve a weight of 150 grams.
3. Confirm that the pH of the PeroxiCare® from Step 2 is ≧10. The pH is determined by taking about <0.1 grams of the toothpaste formulation PeroxiCare® and thoroughly mixing it with ˜20 cc of distilled water contained within a 30 ml Pyrex glass beaker. A drop of this liquid mixture is then added to a strip of pH paper to determine the pH. Allow at least a minute of contact with the pH before comparing the color change to determine the pH. In the unlikely event that the pH is <10, the addition of sodium hydroxide crystals to the toothpaste formulation can be made to achieve a pH level˜10.
4. Add 600 mg of curcumin powder to the mixture from Step 2 and thoroughly mix with a spatula for at 5 minutes to obtain a uniform red mixture.
5. The mixture from Step 4 containing 6 mg/ml of a sodium curcumin salt within a PeroxiCare® mixture is then poured into suitable airless dispensing tubes.

One skilled in the art would understand how to, and be able to, prepare toothpastes containing various amounts of active without undue experimentation.

### Example 18

### The Formation of a Solid Alkali Metal Flavonoid Salt

Primary Objective

The primary objective of this study was to prepare solid alkali metal salts of flavonoids which have the potential of forming soluble alkali metal solution when added to a variety of aqueous solutions.

Sample Preparation

Basis: 1 gram of apigenin

Procedure:
1. Add 1 gram of apigenin powder to an 80 cc “Pyrex” beaker.
2. Add 40 cc of distilled water to the apigenin powder in Step 1 and thoroughly stir.
3. Slowly add NaOH crystals to the mixture of Step 2 while stirring until all the Apigenin powder is dissolved.
4. Heat the soluble apigenin mixture from Step 3 to elevated temperatures slightly in excess of boiling temperatures. It will be observed that the mixture will take on a light brown/reddish color which will darken when the water has completely evaporated.
5. The resulting sodium apigenin salt from Step 4 is set aside and allowed to cool to about <40° C.
6. The sodium apigenin salt from Step 4 is then transferred to a mortar and pestle vessel and then ground to a fine powder.
7. When the sodium apigenin salt is added to a water solution, a yellow-brown soluble alkali metal salt of apigenin results

In a similar manner, the sodium salt of luteolin is prepared by following the stepwise procedures noted for apigenin.

Colorimetric Testing.

Flavonoids and polyphenols actives, including curcuminoids, and the chelates of these chemicals, generally change color when they are converted to an alkali metal salt. This conversion occurs when an active is rinsed with an NaOH solution. The flavones, for example, typically turn a rich yellow color, curcumin turns a reddish brown, tetrahydrocurcumin turns purple, and the chelates turn various colors, as do the other flavonoids, as dictated by their chemical makeup. Our tests use this color change to determine the presence or absence of the active. Generally, the test is performed by washing the surface to be tested with 10% NaOH, or by adding NaOH to a solution that may contain the active. A change in color, either on the surface being tested, or in some or all of the solution being tested, indicates the presence of the active being tested for.

### Example 19

### In Vitro Curcumin and Vanillin Levels in Saliva Comparing Heat and Salt Technologies

Curcumin calcium chelate was fabricated and found to be stable in high pH PeroxiCare® toothpaste. Other curcumin chelates (Boron, Zn) were also found to be stable in this formulation, but were not tested in this study. This study was designed to determine if curcumin solubilized using either a heat solubilization or chelated salt technology performed in the same way as earlier results with the flavone apigenin. Vanillin is a breakdown product of curcumin and would be present if the curcumin had substantially decomposed (the lack of vanillin is indicative of a stable delivery of curcumin).

Eight teeth, four carious and four virgin were separated into four treatment groups based upon the tooth type and compound being tested. Compounds tested were 2% by weight Ca-curcumin chelate in PeroxiCare® (as a salt), and 2% tetrahydro-curcumin heat solubilized in Cremophor/Kolliphor RH-40 in Crest. Additional experiments confirmed that, in general, other curcuminoids in Cremophor/Kolliphor RH-40 in Crest ProHealth behaves similarly to tetrahydro-curcumin.

All teeth were soaked in their respective test paste for four days after which they were washed thoroughly to remove any visible paste or test solution. The salted samples, carious and virgin, then were placed in a first subject's saliva for one hour, removed and placed in new samples of the first subject's saliva for 3 hours and again at 6 and 12 hours, respectively. Analyzing the saliva at each time period provides an elution/leeching profile of the active over time. The same protocol was followed for a second subject. Samples from each time interval were frozen and subsequently subjected to HPLC analysis of the vanillin and curcumin levels present.

TABLE XXV
Vanillin and curcumin concentrations in saliva

| | | Time (h) | | | | | |
|---|---|---|---|---|---|---|---|
| | Tooth | | 0 | 1 | 3 | 6 | 12 |
| Subject | type | | μg/mL | | | | |
| First (2% Ca- | Virgin | Curcumin | 0 | 6.35 | 0.44 | 0.00 | 0.01 |
| Curcumin chelate | | | | | | | |
| salt in PeroxiCare ®) | | | | | | | |
| | | Vanillin | 0 | 0.8 | 0.03 | ND | ND |
| First (2% Ca- | Carious | Curcumin | 0 | 2.65 | 0.75 | 0.18 | N/A |
| Curcumin chelate | | | | | | | |
| salt in PeroxiCare ®) | | | | | | | |
| | | Vanillin | 0 | 0.16 | ND | ND | ND |
| Second (2% | Virgin | Curcumin | 0 | 1.43 | 1.93 | 0.21 | N/A |
| tetrahydrocurcumin | | | | | | | |
| heat solubilized in | | | | | | | |
| Cremophor/Kolliphor | | | | | | | |
| RH-40 in Crest) | | | | | | | |
| | | Vanillin | 0 | 0.10 | ND | ND | N/A |
| Second (2% | Carious | Curcumin | 0 | 4.55 | 2.36 | 0.71 | N/A |
| tetrahydrocurcumin | | | | | | | |
| heat solubilized in | | | | | | | |
| Cremophor/Kolliphor | | | | | | | |
| RH-40 in Crest) | | | | | | | |
| | | Vanillin | 0 | 0.41 | ND | ND | N/A |

It was found that carious teeth seemed to lead to higher concentrations over time suggesting that carious teeth can serve as reservoir of the active. Both technologies had been previously demonstrated to penetrate caries (Example 22).

### Example 20

### In Vitro Apigenin Levels in Saliva Comparing Heat-Solubilized and Salt Technologies

Eight teeth, four carious and four teeth without obvious caries or defects (“virgin” teeth), were separated into four treatment groups. Each treatment group had a particular 2% apigenin compound (either salted in PeroxiCare® or heat solubilized in PS80 in Crest) applied. The treatment was applied for 24 hours and then removed by vigorous washing to remove all surface traces of treatment materials.

The 2% apigenin salt in PeroxiCare® samples, both carious and virgin, then were placed in a first subject's saliva for one hour, removed and placed in another sample of saliva for 3 hours and again at 6 and 12 hours respectively. The same protocol was followed using 2% apigenin heat solubilized in PS80 in Crest using a second subject's saliva. Saliva samples from each time interval were frozen and subsequently HPLC tested to determine the leeched apigenin levels in the saliva.

TABLE XXVI

| | | Time (h) | | | | |
|---|---|---|---|---|---|---|
| | | 0 | 1 | 3 | 6 | 12 |
| Subject | Tooth type | μg/mL | | | | |
| First (2% apigenin salt | Virgin | 0 | 24.6 | 24.6 | 16.0 | 8.6 |
| PeroxiCare ®) | | | | | | |
| First (2% apigenin salt | Carious | — | 21.1 | 16.1 | 18.6 | 4.7 |
| PeroxiCare ®) | | | | | | |
| Second (2% heat | Virgin | 0 | 20.2 | 16.3 | 14.5 | 5.3 |
| solubilized apigenin | | | | | | |
| Crest toothpaste) | | | | | | |
| Second (2% heat | Carious | — | 23.1 | 17.1 | 12.8 | 5.1 |
| solubilized apigenin | | | | | | |
| Crest toothpaste) | | | | | | |

It was found that apigenin leeches into saliva consistently over long intervals for all treatment groups, suggesting that both technologies for apigenin leech active compounds into the oral cavity for at least 12 hours (and perhaps up to 24 hours, as suggested by effectiveness in vivo examples).

When comparing to Example 19, apigenin seems to persist in the oral cavity longer than curcumin and at higher concentrations.

### Example 21

### In Vivo Use Results

A sample of subjects (5) used various concentrations of apigenin salt in Peroxicare toothpaste over extended periods (up to one year) in order to gauge efficacy and tolerability. With a 1% apigenin salt toothpaste (in Peroxicare base), all study participants reported a reduction or elimination of plaque and calculus during regular dental checkups where pre-study dental examinations showed their presence. Part way through the study period, some of the study participants switched to a lower dosage (0.6%) apigenin salt in PeroxiCare® toothpaste. Each of these participants reported the presence of plaque and calculus at their next regularly scheduled dental examination. All study participants reported a reduction in gum inflammation and bleeding while using either formulation.

These results indicate a dose-dependent effectiveness of the active in toothpaste in controlling dental conditions, and appear correlated to the amount of persisted active in saliva based upon dose.

It was hypothesized that the presence of persisted actives in saliva may be from other sources than retention in the saliva. A series of studies was undertaken to determine where the actives were present in teeth during the time the actives were being released into the saliva. One hypothesis was that the actives actually penetrated tooth structures and features, such as cracks, caries, tubules, and at the cementoenamel junction (cej), which is the start of the tooth root where the enamel reaches the cementum. Cracks are common in older patients and for those who have large fillings or grind their teeth. Tubules are exposed in patients with toothbrush abrasion or in areas surrounding caries.

### Example 22

### Tooth Penetration Comparing Active Formulations

In vitro penetration studies were designed to determine the infiltration of the compounds into extracted teeth by considering a number of variables (active polyphenol, toothpaste pH, solubilization technology) in order to determine if one or more compounds penetrated tooth structures in order to contribute to the persistence of the active in saliva during in vivo use.

Teeth, both with and without decay (caries) and with/without dental cracks, were soaked in a toothpaste containing concentrations of at least 1% of the test formulation for 24 hours. The teeth were then sectioned and painted with sodium hydroxide to colorimetrically determine if the test ingredient was present within tooth structures exposed by sectioning.

| | Penetration evident | | | | |
|---|---|---|---|---|---|
| Compound | calculus | caries | cracks | Tubules | Notes |
| Rosocyanin | n/a | no | no | No | |
| (curcumin | | | | | |
| boronate | | | | | |
| [chelate]) | | | | | |
| in water paste | | | | | |
| Apigenin nano | No | no | no | No | Apigenin is |
| powder in | | | | | sparingly |
| water paste | | | | | insoluble |
| | | | | | in water |
| Curcumin | No | no | no | No | Curcumin is |
| powder in | | | | | sparingly |
| water paste | | | | | insoluble |
| | | | | | in water |
| Apigenin and | Yes | yes | yes | yes | |
| Curcumin | | | | | |
| salts in | | | | | |
| PeroxiCare ® | | | | | |
| Apigenin | Yes | yes | yes | Yes | Apigenin salts |
| salts in | | | | | penetrated |
| PeroxiCare ® | | | | | pre-existing |
| | | | | | calculus on |
| | | | | | tooth. |
| Curcumin | n/a | yes | yes | yes | Curcumin is |
| salts in | | | | | unstable in this |
| PeroxiCare ® | | | | | formulation and |
| | | | | | degrades quickly |
| Apigenin PS80 | No | no | no | no | |
| heat | | | | | |
| solubilized | | | | | |
| in Crest | | | | | |
| Curcumin heat | no | yes | no | no | |
| solubilized | | | | | |
| Cremophore/ | | | | | |
| Kolliphor | | | | | |
| RH-40 | | | | | |
| in Crest | | | | | |
| Rosocyanin | n/a | yes | yes | yes | |
| (B-Curcumin | | | | | |
| chelate) salt | | | | | |
| in PeroxiCare ® | | | | | |
| Rosocyanin | n/a | yes | no | no | |
| (B-curcumin | | | | | |
| chelate) heat | | | | | |
| solubilized | | | | | |
| Cremophor/ | | | | | |
| Kolliphor | | | | | |
| RH-40 in Crest | | | | | |
| ProHealth | | | | | |

It was found that salted actives penetrate tooth structures to various degrees, while surfactant solubilized actives generally do not penetrate tooth structures (see table for exceptions). The penetration of tooth structures by these actives was unexpected, and suggests that penetration/leeching of active may be partially responsible for the elevated levels of active observed in saliva over time. In particular, the penetration of pre-existing calculus by apigenin and curcuminoid salts was surprising and supports a hypothesis that penetration is at least part of the cause of the in vivo reports of pre-existing calculus being removed by apigenin salt-based toothpastes. Patients brushing with pastes utilizing either salt or heat solubilized actives also had apigenin present in saliva over time. It is hypothesized that the heat solubilization process creates a muco-adhesive effect. Therefore there may be more than one mechanism causing apigenin to be present in saliva after brushing.

### Example 23

### In Vitro Concentrations of Apigenin Leeched from Teeth

Two teeth, one carious and one virgin (e.g. without obvious fillings, defects or caries) were soaked in a 2% PeroxiCare® apigenin salt toothpaste for 3 days. Two additional teeth, one carious and one virgin were soaked in 2% apigenin PS80 (heat solubilized concentrate) toothpaste for 3 days. The teeth were then washed to remove all visible paste and placed in 4 ml of distilled water. The sample tubes were subjected to 30 minutes of ultrasonic agitation four times during the subsequent 24 hours. Two mls of water was then extracted and frozen. The samples were again subjected to ultrasonic agitation four times during a second 24-hour period, after which the teeth were removed from the water and the samples frozen. The frozen samples were subsequently subjected to HPLC analysis to determine the concentration of the apigenin active in the liquid.

TABLE XXVII

| Sample | Concentration (μg/mL) |
|---|---|
| Salt (post 24 hours) | 36.99 |
| Salt 2/3 (post 48 hours) | 32.05 |
| PS80 (post 24 hours) | 16.45 |
| PS80 2/3 (post 48 hours) | 30.71 |

The experiments indicated that the apigenin active is present in leechate regardless of the technology used to apply the active to the teeth. The results indicate that surface dental structures are at least partially responsible for the persisted presence of the active in vivo.

### Example 24

### In Vivo Penetration of Apigenin in Recently Extracted Teeth

One patient brushed once per day for many months with either 0.5% apigenin salt PeroxiCare® toothpaste (second half of study period) or 1% apigenin salt PeroxiCare® toothpaste (first half of study period) and discovered that two teeth required extraction. The patient was aware of pain in the region but often felt better after brushing and, thus, did not immediately pursue treatment. The patent reported that pain relief continued for up to 12 hours post brushing on the 0.5% apigenin salt toothpaste, and for up to 24 hours post brushing with the 1% apigenin salt toothpaste. The teeth were removed and upon removal were immediately placed, without removal of blood, in separate tubes with 2 ml distilled water. During examination of the extracted teeth, the teeth were removed from the tubes and drops of NaOH solution were put on the teeth to check for the presence of the active compound on the surface of the tooth. The teeth were then sectioned and additional NaOH solution was placed upon the cut surfaces to check for the presence of the active compound in dental structures. Finally, a piece of calculus and pieces of caries were separately scraped off teeth, placed on a pad, and washed with the same NaOH solution. Color changes in the wash solution indicated that the calculus and carious material contained the active.

| Location | Findings | Comments |
|---|---|---|
| Surface | Yes, by color change | Apigenin was found on |
| | | the surface of the |
| | | teeth, and in the |
| | | NaOH wash solution. |
| In Carious | Yes, by color change | Apigenin was found on |
| Lesions | in carious lesion area | the surface of the |
| | of sectioned tooth | teeth, and in the |
| | Yes, by leeching of | NaOH wash solution. |
| | active from extracted | |
| | carious material into | |
| | wash fluids. | |
| Penetrates | Yes, by color change | |
| plaque | of plaque/film on | |
| | teeth when washed | |
| | with NaOH. | |
| Penetrates | Yes, by color change | Apigenin was found in |
| calculus | | the NaOH wash |
| | | solution. |
| | | Confirmed by further |
| | | in vitro studies. |
| Penetrates | No, by lack of color | |
| internal tooth | change in sectioned | |
| structures | tooth structures | |

It is noted that the patient documented over the course of the study dose-dependent results for both pain relief and removal of calculus, with the 0.5% apigenin salt PeroxiCare® toothpaste being noticeably less effective than the 1% apigenin salt PeroxiCare® toothpaste (no calculus or pain with the 1% compound, some calculus and pain returning with the 0.5% compound).

This work supports our earlier findings of apigenin in saliva following chronic use and apigenin salts in vitro penetrating decay, plaque, and calculus, leeching and being present in saliva for extended periods of time after application.

### Example 25

### Tooth Penetration in Paste after 24 Hours or Solution after 30 Minutes

A series of tests were performed to isolate the delivery vehicle from the active in order to determine whether the active compound was responsible for penetration of dental structures (or if the carrier was responsible for the penetration). Toothpaste compounds using alkali metal salt actives and heat solubilized actives (compounded as described above) were applied to extracted teeth for a period of time. After application, the teeth were washed to remove external traces of the active, sectioned, and the sectioned pieces washed with NaOH to colorimetrically check for the presence of the active within tooth structures. Table XXVIII details the results of this experiment.

TABLE XXVIII

| | Toothpaste | Solution |
|---|---|---|
| | Penetration of | Penetration of |
| | dentinal tubules | dentinal tubules |
| | at 1 day | at 30 minutes |
| APIGENIN | | |
| Salt | Yes | Yes |
| Ps80 | No | No |
| CURCUMIN | | |
| Salt | Yes | Maybe - Crack |
| | | definitely |
| Cremophor | No | No |
| Boron Chelate of Curcumin | | |
| (rosocyanine) | | |
| Salt | Yes | Not done |
| Cremophor/Kolliphor RH-40 | Not done | Not done |
| ECGC | | |
| Salt | No | No |
| Ps80 | No | No |
| Powder | No | No |
| RESVERATROL | | |
| Salt | No | No |
| Powder | No | No |
| QUERCETIN | | |
| Salt | Yes | Not Done |
| Ps80 | Not done | Not Done |
| CA CURCUMIN CHELATE | | |
| Salt | Yes | Yes - 1 minute |
| Cremophor (heat) | No | No |
| TETRAHYDRO CURCUMIN | | |
| Salt | Maybe | Not done |
| Cremophor (heat) | No | No |

### Example 26

### Synthesis of Calcium-Curcumin Chelate

Step 1. 36.8 g curcumin (0.1 mole) was heated with stirring at 40-50° C. in a 50:50 mixture of methanol and acetone (400 mL) under nitrogen for about 4 hours to obtain a clear solution.

Step 2. The solution was allowed to cool to ambient temperature.

Step 3. A solution of calcium chloride (5.6 g, 0.05 mole) in 100 mL methanol was then added to the solution of curcumin, and stirred at ambient temperature for 30 minutes.

Step 4. The pH of this solution was raised and adjusted to 8.75-9.0 by slowly adding solution of ammonia in methanol (NH₃/CH₃OH). A deep orange red precipitate was formed that was filtered.

Step 5. The red solids obtained were suspended in water and stirred to remove soluble byproduct ammonium chloride, The remaining red orange solids were washed by suspending and stirring in methanol, and filtered and dried to obtain red orange calcium-curcumin chelate (about 20 g).

The presence of calcium was tested by suspending the solids in acetic acid and stirring. Bright yellow solids appeared giving indication of reversal to starting curcumin.

### Example 27

### Synthesis of Zinc-Curcumin Chelate

Step 1. 36.8 g curcumin (0.1 mole) was heated with stirring at 40-50° C. in a 50:50 mixture of methanol and acetone (400 mL) under nitrogen for about 4 hours to obtain a clear solution.

Step 2. The solution was allowed to cool to ambient temperature. A solution of zinc chloride (6.87 g, 0.0525 mole) in 200 mL methanol was then added to the solution of curcumin, and stirred at ambient temperature for 30 minutes.

Step 3. The pH of this solution was raised and adjusted to 8.75-9.0 by slowly adding solution of ammonia in methanol (NH₃/CH₃OH). A deep orange red precipitate was formed that was filtered.

Step 4. The red solids obtained were suspended in water and stirred to remove soluble byproduct ammonium chloride, The remaining red orange solids were washed by suspending and stirring in methanol, and filtered and dried to obtain orange zinc-curcumin chelate about 13 g.

The presence of zinc was tested by suspending the solids in acetic acid and stirring. Bright yellow solids appeared giving indication of reversal to starting curcumin.

### Example 28

### Preparation of Apigenin/PS 80—Peroxicare® Toothpaste

This method of preparation provides an alternative formulation method for an active solubilized in a surfactant using the heat-methodologies described above. This technique is applicable to flavonoids and curcuminoids solubilized in PS80.

Step 1. 40 g of PeroxiCare® toothpaste was mixed thoroughly with 10 g apigenin—PS 80 concentrate solution (about 4% apigenin).

Step 2. Agitate mixture for 30 minutes to obtain yellow 1% apigenin containing toothpaste.

One skilled in the art would understand how to, and be able to, prepare toothpastes containing various amounts of active, up to approximately 2% concentrations, without undue experimentation.

The viscosity of this alkaline toothpaste was slightly lower than the PeroxiCare® toothpaste as observed by flow.

### Example 29

### Preparation of Apigenin/PS 80—Crest Prohealth Toothpaste

This method of preparation provides an alternative formulation method for a toothpaste utilizing apigenin solubilized in a surfactant using the heat-methodologies described above. This technique is applicable to flavonoids and curcuminoids solubilized in PS80. These techniques were also utilized with other acidic toothpastes, such as “original” Crest, by substituting the toothpaste base. All other conditions remain the same.

Step 1. 40 g of ProHealth toothpaste was mixed thoroughly with 10 g apigenin—PS 80 concentrate solution (about 4% apigenin).

Step 2. Agitate mixture for 30 minutes containing about 1% apigenin containing toothpaste.

One skilled in the art would understand how to, and be able to, prepare toothpastes containing various amounts of active, up to approximately 2% concentrations, without undue experimentation.

The viscosity of this acidic toothpaste was slightly lower than the ProHealth toothpaste as observed by flow.

### Example 30

### Preparation of Apigenin-Curcumin/PS 80—Peroxicare® Toothpaste

Step 1. 40 g of PeroxiCare® toothpaste was mixed thoroughly with 10 g apigenin/curcumin—PS 80 solution (about 4% apigenin/10% curcumin)

Step 2. Agitate mixture for 30 minutes to obtain deep orange—red 1% apigenin/2.5% curcumin containing toothpaste.

The viscosity of this alkaline toothpaste was slightly lower than the PeroxiCare® toothpaste as observed by flow.

One skilled in the art would understand how to, and be able to, prepare toothpastes containing various amounts of active without undue experimentation.

It should be understood that a wide range of changes and modifications could be made to the embodiments described above. It is therefore intended that the foregoing description illustrates rather than limits this invention, and that it is the following claims, including all equivalents, which define this invention.

All references cited in the present specification are hereby incorporated by reference in their respective entireties.

While the invention has been described with reference to an exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to any particular embodiment disclosed as the best mode contemplated for carrying out this invention, but that the invention will include all embodiments falling within the scope of the applied claims.

What is claimed is:

1. A multi-cavity dispensing container for delivering a polyphenol and a toothpaste comprising:
a first cavity containing a toothpaste or gel, and
a second cavity containing a gel or paste containing a polyphenol.

2. A multi-cavity dispensing container as in claim 1 wherein the polyphenol is a flavonoid and the flavonoid is in the form of a concentrate.

3. A solid alkaline metal flavonoid salt.

4. A solid alkaline metal flavonoid salt as in claim 3, wherein the flavonoid is apigenin and the salt is in the form of a powder.

5. A method of preventing or treating disorders or diseases of the oral cavity, comprising:
administering a composition comprising:
a solubilized flavonoid, solubilized curcuminoid, or combinations thereof;
whereby said solubilized flavonoid, solubilized curcuminoid, or combinations thereof persist in said oral cavity longer than nonsolubilized flavonoid, nonsolubilized curcuminoid, or combinations thereof.

6. The method of claim 5, wherein the concentrations of said solubilized flavonoid, solubilized curcuminoid, or combinations thereof are selected from the group consisting of 0.5%-4.0%, 1.0%-2.0%, and 0.1% to 20%.

7. The method of claim 5, wherein said solubilized flavonoid is selected from the group consisting of apigenin, luteolin, kaemferol, quercetin, myricetin, daidzein, genistein, catechins, gallocatechins, naringin, rutin, hesperitin, anthocyanidins, and combinations thereof.

8. The method of claim 5, wherein said solubilized curcuminoid is selected from the group consisting of curcumin, tetrahydro-curcumin, and combinations thereof.

9. The method of claim 7, wherein said solubilized flavonoid is apigenin.

10. The method of claim 8, wherein said solubilized curcuminoid is curcumin.

11. The method of claim 5, wherein said disorders or diseases of the oral cavity are selected from the group consisting of dental plaque, dental caries, periodontal disease, oral cancer, oral chemotherapy sequelae, gingivitis, herpetic lesions, cold sores, aphthous ulcers, dry mouth, toothache, wound, tooth sensitivity or pain, denture stomatitis, fungal, viral or bacterial infections, and combinations thereof.

12. The method of claim 5, wherein said solubilized flavonoid, solubilized curcuminoid, or combinations thereof persist in said oral cavity for up to at least twelve hours.

13. The method of claim 5, wherein said solubilized flavonoid, solubilized curcuminoid, or combinations thereof persist in said oral cavity for up to at least six hours.

14. The method of claim 5, wherein said solubilized flavonoid, solubilized curcuminoid, or combinations thereof persist in said oral cavity for up to at least three hours.

15. The method of claim 5, wherein said solubilized flavonoid, solubilized curcuminoid, or combinations thereof penetrate a dental structure.

16. The method of claim 15, wherein said dental structure is selected from the group consisting of plaque, tartar, calculus, caries, cracks, dentinal tubules, sulcus, and cementoenamel junctions.

17. The method of claim 5, wherein said solubilized flavonoid and/or solubilized curcuminoid is prepared by the group consisting of chelation, heat solubilized concentration, and alkali metal salting, and combinations thereof.

18. A composition, comprising a chelated flavonoid or chelated curcuminoid, and an orally-acceptable carrier.

19. The composition of claim 18, wherein said chelated flavonoid or chelated curcuminoid comprises an alkali metal selected from the group consisting of calcium, zinc, magnesium, iron, copper, and boron.

20. The composition of claim 18, wherein active flavonoid or active curcuminoid is released from said chelated flavonoid or chelated curcuminoid upon acidification of said composition.

21. The composition of claim 20, wherein said acidification is selected from the group consisting of reducing the acidity to pH 4, reducing the acidity to pH 3, and reducing the acidity to pH 2.
